(19) United States
(12) Patent Application Publication
(10) Pub. No.: US 2026/0269013 A1
(43) Pub. Date: Sep. 10, 2026

(54) Health Analysis Based on Identity-by-Descent Segments

(71) Applicant: 23andMe Genomics LLC, Los Altos, CA (US)

(72) Inventors: Adityasai Ambati, Dublin, CA (US); Anna Bao Zhen Guan, Los Altos, CA (US); Jingran Wen, Los Altos, CA (US); David A. Hinds, Los Altos, CA (US); Bertram Koelsch, Salt Lake City, UT (US); William Allen Freyman, Oakland, CA (US)

(21) Appl. No.: 19/559,832

(22) Filed: Mar. 6, 2026

Related U.S. Application Data
(60) Provisional application No. 63/769,383, filed on Mar. 10, 2025.

(51) Int. Cl. G16B 30/10; G16B 5/00; G16B 20/20; G16B 40/20; G16H 20/00; G16H 50/70

(52) U.S. Cl. CPC G16B 30/10; G16B 5/00; G16B 20/20; G16B 40/20; G16H 20/00; G16H 50/70

(57) ABSTRACT

An embodiment may involve: obtaining phased exome sequence data for a genomic region, wherein the genomic region contains a pathogenic variant site; obtaining statistically phased sequence data for the genomic region; traversing the genomic region from the pathogenic variant site in an upstream direction until a first non-missing heterozygous site is present in both sequence data; based on haplotypes of the first non-missing heterozygous site from the sequence data, determining an upstream phase; traversing the genomic region from the pathogenic variant site in a downstream direction until a second non-missing heterozygous site is present in both sequence data; based on haplotypes of the second non-missing heterozygous site from the sequence data, determining a downstream phase; and in response to determining that the upstream phase and the downstream phase are both a particular phase, assigning the particular phase to the genomic region.

### CROSS-REFERENCE TO RELATED APPLICATIONS

The present application claims the benefit of U.S. Provisional Application No. 63/769,383, filed Mar. 10, 2025. The contents of which are hereby incorporated by reference in their entirety.

### BACKGROUND

Genetic analysis allows for the early prediction of diseases by identifying specific genetic markers associated with various conditions, enabling proactive medical interventions and personalized treatment plans. Conducting this analysis efficiently on a computing system enhances its accessibility, speed, and accuracy, as algorithms can rapidly process vast genetic datasets to detect patterns that might be impossible for humans to otherwise discern. However, these algorithms benefit from being computationally efficient, as those that require fewer computing resources can supply results more quickly while using less processor, memory, network, and/or power capacity. Such computational efficiency also allows for real-time assessment, ultimately improving healthcare outcomes by enabling earlier diagnoses and targeted therapies.

### SUMMARY

Example embodiments described herein include techniques for determining a phasing for a genomic region in one dataset (e.g., a dataset containing genotype data for only a certain number of genes) based on the phasing of a pathogenic variant site from a different dataset (e.g., a dataset containing whole exome sequencing). By determining the phasing for the genomic region, the presence of the pathogenic variant site (or separate pathogenic variant sites) can be readily identified within the genomic region through the identification of identity-by-descent (IBD) segments for other individuals that have the pathogenic variant site (and/or the separate pathogenic variant sites). In this way, the presence of pathogenic variants may be imputed even for individuals for whom whole exome sequencing has not been performed. This can lead to improved diagnoses and, even more importantly, targeted treatments (e.g., prophylactic treatments that reduce the risk of contracting a disease associated with an imputed pathogenic variant or delays disease onset of a disease associated with an imputed pathogenic variant).

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by a data processing apparatus, cause the apparatus to perform the actions.

In a first aspect, a computer-implemented method is provided. The computer-implemented method includes obtaining phased exome sequence data for a genomic region. The genomic region contains a pathogenic variant site. The computer-implemented method also includes obtaining statistically phased sequence data for the genomic region. Additionally, the computer-implemented method includes traversing the genomic region from the pathogenic variant site in an upstream direction until a first non-missing heterozygous site is present in both the phased exome sequence data and the statistically phased sequence data. Further, the computer-implemented method includes, based on haplotypes of the first non-missing heterozygous site from the phased exome sequence data and the statistically phased sequence data, determining an upstream phase. In addition, the computer-implemented method includes traversing the genomic region from the pathogenic variant site in a downstream direction until a second non-missing heterozygous site is present in both the phased exome sequence data and the statistically phased sequence data. Still further, the computer-implemented method includes, based on haplotypes of the second non-missing heterozygous site from the phased exome sequence data and the statistically phased sequence data, determining a downstream phase. Even further, the computer-implemented method includes, in response to determining that the upstream phase and the downstream phase are both a particular phase, assigning the particular phase to the genomic region.

In a second aspect, a non-transitory, computer-readable medium having instructions stored thereon is provided. The instructions, when executed by a computing system, cause the computing system to perform a method. The method includes obtaining phased exome sequence data for a genomic region. The genomic region contains a pathogenic variant site. The method also includes obtaining statistically phased sequence data for the genomic region. Additionally, the method includes traversing the genomic region from the pathogenic variant site in an upstream direction until a first non-missing heterozygous site is present in both the phased exome sequence data and the statistically phased sequence data. Further, the method includes, based on haplotypes of the first non-missing heterozygous site from the phased exome sequence data and the statistically phased sequence data, determining an upstream phase. In addition, the method includes traversing the genomic region from the pathogenic variant site in a downstream direction until a second non-missing heterozygous site is present in both the phased exome sequence data and the statistically phased sequence data. Still further, the method includes, based on haplotypes of the second non-missing heterozygous site from the phased exome sequence data and the statistically phased sequence data, determining a downstream phase. Even further, the method includes, in response to determining that the upstream phase and the downstream phase are both a particular phase, assigning the particular phase to the genomic region.

In a third aspect, a system is provided. The system includes one or more processors. The system also includes a memory, containing program instructions that, upon execution by the one or more processors, cause the system to perform a method. The method includes obtaining phased exome sequence data for a genomic region. The genomic region contains a pathogenic variant site. The method also includes obtaining statistically phased sequence data for the genomic region. Additionally, the method includes traversing the genomic region from the pathogenic variant site in an upstream direction until a first non-missing heterozygous site is present in both the phased exome sequence data and the statistically phased sequence data. Further, the method includes, based on haplotypes of the first non-missing heterozygous site from the phased exome sequence data and the statistically phased sequence data, determining an upstream phase. In addition, the method includes traversing the genomic region from the pathogenic variant site in a downstream direction until a second non-missing heterozygous site is present in both the phased exome sequence data and the statistically phased sequence data. Still further, the method includes, based on haplotypes of the second non-missing heterozygous site from the phased exome sequence data and the statistically phased sequence data, determining a downstream phase. Even further, the method includes, in response to determining that the upstream phase and the downstream phase are both a particular phase, assigning the particular phase to the genomic region.

These, as well as other embodiments, aspects, advantages, and alternatives, will become apparent to those of ordinary skill in the art by reading the following detailed description, with reference where appropriate to the accompanying drawings. Further, this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE DRAWINGS

FIG. 1 illustrates a schematic drawing of a computing device, in accordance with example embodiments.

FIG. 2A illustrates a schematic drawing of a server device cluster, in accordance with example embodiments.

FIG. 2B illustrates a method for training a machine-learned model, in accordance with example embodiments.

FIG. 2C illustrates a method of making a prediction using a machine-learned model, in accordance with example embodiments.

FIG. 3 depicts imputation of genotypes based on IBD segments, in accordance with example embodiments.

FIG. 4A depicts identification of pathogenic alleles based on IBD segments, in accordance with example embodiments.

FIG. 4B depicts the training of a machine-learned model used to identify pathogenic alleles based on IBD segments, in accordance with example embodiments.

FIG. 5 depicts a comparison between direct exome sequence-based phasing and statistical phasing, in accordance with example embodiments.

FIG. 6 depicts a phase inference method, in accordance with example embodiments.

FIG. 7A depicts an instance phase inference, in accordance with example embodiments.

FIG. 7B depicts an instance phase inference, in accordance with example embodiments.

FIG. 7C depicts an instance phase inference, in accordance with example embodiments.

FIG. 8A depicts an IBD segment in relation to a genomic region and a pathogenic variant site, in accordance with example embodiments.

FIG. 8B depicts a sine-transformed position of a variant, given start and end of IBD segments, in accordance with example embodiments.

FIG. 9 depicts how the sine transformation can identify false positives, in accordance with example embodiments.

FIG. 10 depicts parameters and results of regression models, in accordance with example embodiments.

FIG. 11 is a flow chart, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless stated as such. Thus, other embodiments can be utilized and other changes can be made without departing from the scope of the subject matter presented herein. Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations. For example, the separation of software features into “client” and “server” components may occur in a number of ways.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

Unless clearly indicated otherwise herein, the term “or” is to be interpreted as the inclusive disjunction. For example, the phrase “A, B, or C” is true if any one or more of the arguments A, B, C are true, and is only false if all of A, B, and C are false.

### I. DEFINITIONS

The following definitions are provided to clarify terms as they are used in this application. These definitions are intended to aid in understanding the invention but should not be considered limiting. Unless otherwise specified, terms should be interpreted in a manner consistent with their ordinary meaning in the relevant technical field. In cases where a term has multiple meanings, the interpretation most applicable to this disclosure should be used.

A genome is the complete set of genetic material present in an organism, including all of its deoxyribonucleic acid (DNA) and/or ribonucleic acid (RNA). It includes the full sequence of nucleotides that encode the instructions necessary for the growth, development, functioning, and reproduction of the organism. The genome includes both coding regions, which contain genes responsible for protein synthesis, and non-coding regions, which may regulate gene expression and other cellular functions.

An exome is the portion of a genome that consists of all exons, which are the protein-coding regions of genes. It includes the sequences that are transcribed into messenger RNA (mRNA) and subsequently translated into proteins, excluding non-coding introns and intergenic regions. While the exome represents only a small fraction of the entire genome, it contains the vast majority of known disease-associated genetic variants, making it a focus in genetic analysis, diagnostics, and therapeutic development. In the context of this application, the term “exome” encompasses both the complete set of exon sequences and any relevant variations or modifications.

Identical-by-descent (IBD) segments are continuous stretches of DNA that are inherited from a common ancestor without recombination, meaning they are identical between two or more individuals due to shared ancestry. These segments can be used to determine genetic relatedness, reconstruct pedigrees, and identify disease-associated genetic variants. IBD segments can be identified through computational analysis of genomic data, comparing haplotypes across individuals to detect regions of high similarity.

A haplotype is a specific combination of genetic variants, such as single nucleotide polymorphisms (SNPs) or other markers that are inherited together on the same chromosome from a single parent. Haplotypes can provide valuable information about genetic ancestry, population structure, and disease risk by identifying regions of the genome that are shared among individuals or populations. Because recombination tends to preserve these sequences over generations, haplotypes serve as useful markers in genetic association studies and inheritance analysis.

Diploid cells, in humans, contain two complete sets of chromosomes (46 total), with one set inherited from each parent. This diploid state is present in most somatic (body) cells and enables genetic recombination, variation, and proper development. In contrast, human haploid cells, such as sperm and egg cells, contain only a single set of chromosomes (23 total). During fertilization, two haploid gametes combine to restore the diploid number in the resulting zygote, providing genetic continuity across generations.

Phasing is the process of determining the parental origin of alleles in a diploid organism by reconstructing haplotypes from genotype data. This technique assigns genetic variants to specific chromosomes inherited from each parent, resolving whether two alleles at a given locus are on the same chromosome or different ones. Phasing can be used for identifying IBD segments, analyzing recombination events, improving the accuracy of genetic association studies, and enabling personalized medicine approaches. In the context of this application, “phasing” refers to methods, algorithms, and techniques used to infer haplotypes from genotypic data for research, clinical, and biotechnological purposes.

A phasing panel can be a reference dataset consisting of haplotypes derived from a population or a specific group of individuals, used to infer the phase of genotyped alleles in genetic analysis. This panel aids in resolving the arrangement of alleles on individual chromosomes by comparing an individual's genotype data to known haplotype structures, improving the accuracy of genetic phasing.

A proband is the individual in a family or genetic study who is first identified as having a particular trait, condition, or disease, serving as the starting point for genetic analysis. In medical genetics, the proband is typically the first affected family member to be clinically evaluated, and their genetic data is used to investigate inheritance patterns, familial risk, and potential genetic variants associated with the condition. Herein, the term “proband” may further refer to a reference individual used as the basis of a genetic analysis.

The variant call format (VCF) is a standard structure for storing variant data. VCF is a text file format (often stored in a compressed manner). It contains meta-information lines, a header line, and then data lines each containing information about a position in the genome. BCF, or the binary variant call format, is the binary version of VCF. It keeps the same information in VCF, but is much more efficient to process especially for large sets of samples.

### II. EXAMPLE COMPUTING DEVICES AND CLOUD-BASED COMPUTING ENVIRONMENTS

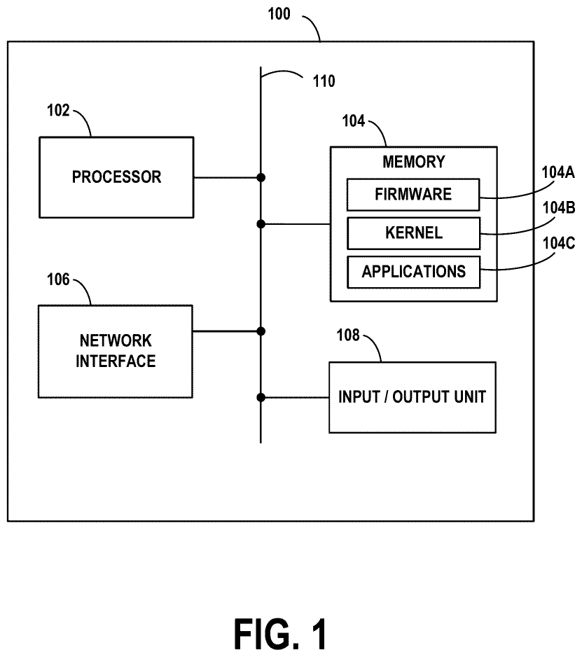

FIG. 1 is a simplified block diagram exemplifying a computing device 100, illustrating some of the components that could be included in a computing device arranged to operate in accordance with the embodiments herein. Computing device 100 could be a client device (e.g., a device actively operated by a user), a server device (e.g., a device that provides computational services to client devices), or some other type of computational platform. Some server devices may operate as client devices from time to time in order to perform particular operations, and some client devices may incorporate server features.

In this example, computing device 100 includes processor 102, memory 104, network interface 106, and input/output unit 108, all of which may be coupled by system bus 110 or a similar mechanism. In some embodiments, computing device 100 may include other components and/or peripheral devices (e.g., detachable storage, printers, and so on).

Processor 102 may be one or more of any type of computer processing element, such as a central processing unit (CPU), a graphical processing unit (GPU), a digital signal processor (DSP), a network processor, an encryption processor, and/or a form of integrated circuit or controller that performs processor operations. In some cases, processor 102 may be one or more single-core processors. In other cases, processor 102 may be one or more multi-core processors with multiple independent processing units. Processor 102 may also include register memory for temporarily storing instructions being executed and related data, as well as cache memory for temporarily storing recently used instructions and data.

GPUs, in particular, have grown in importance. They include specialized circuitry designed to perform rapid mathematical calculations for rendering graphics, processing large datasets, and supporting machine learning. A GPU typically consists of hundreds or thousands of small cores that operate simultaneously, facilitating the decomposition of tasks into smaller, more manageable pieces that are processed in parallel. This parallelism allows GPUs to be significantly faster than traditional CPUs for certain types of calculations.

Memory 104 may be any form of computer-usable memory, including but not limited to random access memory (RAM), read-only memory (ROM), and non-volatile memory (e.g., flash memory, hard disk drives, solid state drives, compact discs (CDs), digital video discs (DVDs), and/or tape storage). Thus, memory 104 represents both main memory units, as well as long-term storage. Herein, any non-volatile memory may be referred to as persistent storage.

Memory 104 may store program instructions and/or data on which program instructions may operate. By way of example, memory 104 may store these program instructions on a non-transitory, computer-readable medium, such that the instructions are executable by processor 102 to carry out any of the methods, processes, or operations disclosed in this specification or the accompanying drawings.

As shown in FIG. 1, memory 104 may include firmware 104A, kernel 104B, and/or applications 104C. Firmware 104A may be program code used to boot or otherwise initiate some or all of computing device 100. Kernel 104B may be an operating system, including modules for memory management, scheduling and management of processes, input/output, and communication. Kernel 104B may also include device drivers that allow the operating system to communicate with the hardware modules (e.g., memory units, networking interfaces, ports, and buses) of computing device 100. Applications 104C may be one or more user-space software programs, such as web browsers or email clients, as well as any software libraries used by these programs. Memory 104 may also store data used by these and other programs and applications.

Network interface 106 may take the form of one or more wireline interfaces, such as Ethernet (e.g., Fast Ethernet, Gigabit Ethernet, 10 Gigabit Ethernet, Ethernet over fiber, and so on). Network interface 106 may also support communication over one or more non-Ethernet media, such as coaxial cables or power lines, or over wide-area media, such as Synchronous Optical Networking (SONET), Synchronous Digital Hierarchy (SDH), Data Over Cable Service Interface Specification (DOCSIS), or other technologies. Network interface 106 may additionally take the form of one or more wireless interfaces, such as IEEE 802.11 (Wifi), BLUETOOTH®, global positioning system (GPS), or a wide-area wireless interface. However, other forms of physical layer interfaces and other types of standard or proprietary communication protocols may be used over network interface 106. Furthermore, network interface 106 may comprise multiple physical interfaces. For instance, some embodiments of computing device 100 may include Ethernet, BLUETOOTH®, and Wifi interfaces.

Input/output unit 108 may facilitate user and peripheral device interaction with computing device 100. Input/output unit 108 may include one or more types of input devices, such as a keyboard, a mouse, a touch screen, and so on. Similarly, input/output unit 108 may include one or more types of output devices, such as a screen, monitor, printer, and/or one or more light emitting diodes (LEDs). Additionally or alternatively, computing device 100 may communicate with other devices using a universal serial bus (USB) or high-definition multimedia interface (HDMI) port interface, for example.

In some embodiments, one or more computing devices like computing device 100 may be deployed. The exact physical location, connectivity, and configuration of these computing devices may be unknown and/or unimportant to client devices. Accordingly, the computing devices may be referred to as “cloud-based” devices that may be housed at various remote data center locations.

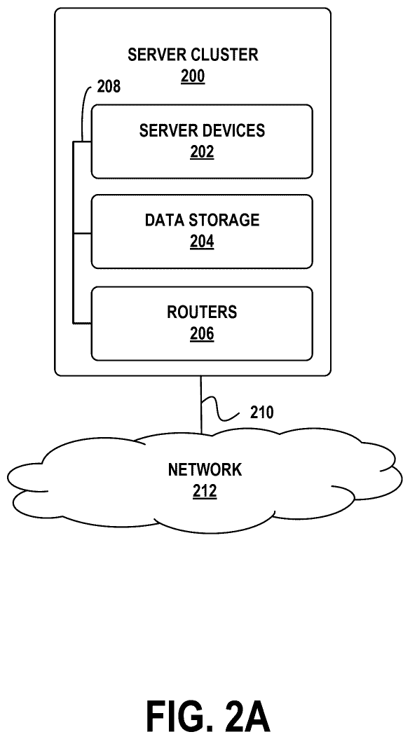

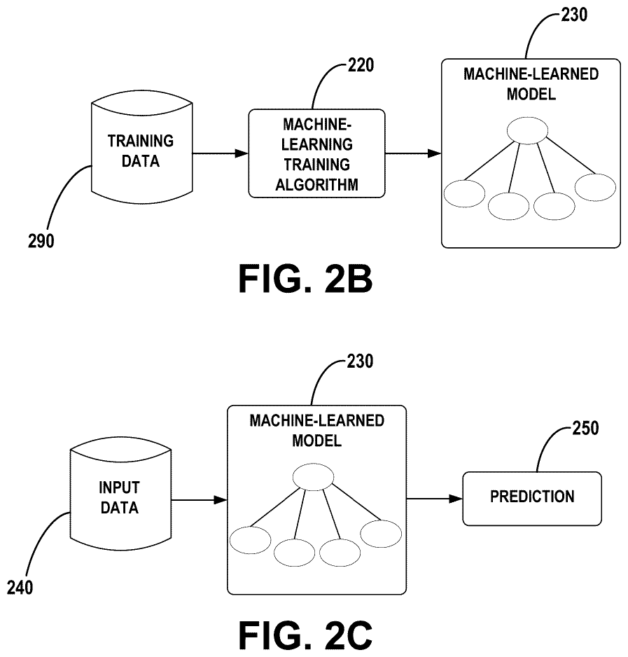

FIG. 2A depicts a cloud-based server cluster 200 in accordance with example embodiments. In FIG. 2A, operations of a computing device (e.g., computing device 100) may be distributed between server devices 202, data storage 204, and routers 206, all of which may be connected by local cluster network 208. The number of server devices 202, data storages 204, and routers 206 in server cluster 200 may depend on the computing task(s) and/or applications assigned to server cluster 200.

For example, server devices 202 can be configured to perform various computing tasks of computing device 100. Thus, computing tasks can be distributed among one or more of server devices 202. To the extent that these computing tasks can be performed in parallel, such a distribution of tasks may reduce the total time to complete these tasks and return a result. For purposes of simplicity, both server cluster 200 and individual server devices 202 may be referred to as a “server device.” This nomenclature should be understood to imply that one or more distinct server devices, data storage devices, and cluster routers may be involved in server device operations.

Data storage 204 may be data storage arrays that include drive array controllers configured to manage read and write access to groups of hard disk drives and/or solid state drives. The drive array controllers, alone or in conjunction with server devices 202, may also be configured to manage backup or redundant copies of the data stored in data storage 204 to protect against drive failures or other types of failures that prevent one or more of server devices 202 from accessing units of data storage 204. Other types of memory aside from drives may be used.

Routers 206 may include networking equipment configured to provide internal and external communications for server cluster 200. For example, routers 206 may include one or more packet-switching and/or routing devices (including switches and/or gateways) configured to provide (i) network communications between server devices 202 and data storage 204 via local cluster network 208, and/or (ii) network communications between server cluster 200 and other devices via communication link 210 to network 212.

Additionally, the configuration of routers 206 can be based at least in part on the data communication requirements of server devices 202 and data storage 204, the latency and throughput of the local cluster network 208, the latency, throughput, and cost of communication link 210, and/or other factors that may contribute to the cost, speed, fault-tolerance, resiliency, efficiency, and/or other design goals of the system architecture.

As a possible example, data storage 204 may include any form of database, such as a structured query language (SQL) database or a No-SQL database (e.g., MongoDB). Various types of data structures may store the information in such a database, including but not limited to files, tables, arrays, lists, trees, and tuples. Furthermore, any databases in data storage 204 may be monolithic or distributed across multiple physical devices.

Server devices 202 may be configured to transmit data to and receive data from data storage 204. This transmission and retrieval may take the form of SQL queries or other types of database queries, and the output of such queries, respectively. Additional text, images, video, and/or audio may be included as well. Furthermore, server devices 202 may organize the received data into web page or web application representations. Such a representation may take the form of a markup language, such as HTML, XML, JSON, or some other standardized or proprietary format. Moreover, server devices 202 may have the capability of executing various types of computerized scripting languages, such as but not limited to Perl, Python, PHP Hypertext Preprocessor (PHP), Active Server Pages (ASP), JAVASCRIPT®, and so on. Computer program code written in these languages may facilitate the providing of web pages to client devices, as well as client device interaction with the web pages. Alternatively or additionally, JAVA® may be used to facilitate generation of web pages and/or to provide web application functionality.

FIG. 2B illustrates a method of training a machine-learned model 230 (e.g., an artificial neural network), according to example embodiments. The method of FIG. 2B may be performed by a device (e.g., the computing device 100 illustrated in FIG. 1), in some embodiments. As illustrated, the machine-learned model 230 may be trained using a machine-learning training algorithm 220 based on training data 290 (e.g., based on patterns within the training data 290). While only one machine-learned model 230 is illustrated in FIGS. 2B and 2C, it is understood that multiple machine-learned models could be trained simultaneously and/or sequentially and used to perform the predictions described herein. Ultimately, a prediction 250 may be made using the trained machine-learned model 230.

The machine-learned model 230 may include, but is not limited to: an artificial neural network (e.g., a convolutional neural network (CNN), a generative adversarial network (GAN), a recurrent neural network, a Bayesian network, a hidden Markov model, a Markov decision process, a logistic regression function, a suitable statistical machine-learning algorithm, and/or a heuristic machine-learning system), a support vector machine, a regression tree, an ensemble of regression trees (also referred to as a regression forest), a decision tree, an ensemble of decision trees (also referred to as a decision forest), or some other machine-learning model architecture or combination of architectures. The machine-learning training algorithm 220 may involve supervised learning, semi-supervised learning, reinforcement learning, and/or unsupervised learning. Similarly, the training data 290 may include labeled training data and/or unlabeled training data.

Using the training data 290, the machine-learning training algorithm 220 may attempt to make a prediction. If the predicted outcome for the input piece of training data 290 matches the label ascribed to the training data 290, this may reinforce the machine-learned model 230 being developed by the machine-learning training algorithm 220. If the predicted outcome for the input piece of training data 290 does not match the label ascribed to the training data 290, the machine-learned model 230 being developed by the machine-learning training algorithm 220 may be modified to accommodate the difference (e.g., the weight of a given factor within the artificial neural network of the machine-learned model 230 may be adjusted). Additionally or alternatively, in some embodiments, the machine-learning training algorithm 220 may enforce additional rules during the training of the machine-learned model 230 (e.g., by setting and/or adjusting one or more hyperparameters).

Once the machine-learned model 230 is trained by the machine-learning training algorithm 220 (e.g., using the method of FIG. 2B), the machine-learned model 230 may be used to make one or more predictions. For example, a device (e.g., the computing device 100 illustrated and described with reference to FIG. 1), may make a prediction 250 using the machine-learned model 230 based on input data 240, as illustrated in FIG. 2C.

As illustrated in FIG. 2C, the machine-learned model 230 can receive input data 240 and generate and output one or more predictions 250 about input data 240. For example, the machine-learned model 230 may include a classifier used to classify objects into different groups or categories. In such cases, the prediction 250 may represent the group or category to which the machine-learned model 230 classified the input data 240.

While the same device (e.g., the computing device 100 shown and described with reference to FIG. 1) may be used to both train the machine-learned model 230 (e.g., as illustrated in FIG. 2B) and make use of the machine-learned model 230 to make a prediction 250 (e.g., as illustrated in FIG. 2C), this need not be the case. In some embodiments, for example, a computing device may execute the machine-learning training algorithm 220 to train the machine-learned model 230 and may then transmit the machine-learned model 230 to another computing device for use in making one or more predictions 250. In the context of this disclosure, for example, a computing device may be used to initially train the machine-learned model 230 and then this machine-learned model 230 could be stored for later use. For example, the machine-learned model 230 could be trained and then incorporated into a genetic services platform for future use.

### III. IDENTIFYING CANDIDATES FOR HEALTH ANALYSIS

A sampling of related individuals can be leveraged to find genomic segments that are shared due to common ancestry. These individuals share IBD segments that can be used to infer non-genotyped pathogenic variants harbored within them. These non-genotyped pathogenic variants may be disease-associated genetic mutations that are not directly observed or measured in a genetic assay due to limitations in genotyping technology, coverage gaps, or the use of genotyping arrays that only capture a subset of known variants.

Since pathogenic variants are shared if present in a common ancestor, IBD segments can be used to identify likely carriers of these variants. An approach uses already sequenced whole exomes of a large number of individuals (e.g., a database of thousands or tens of thousands of individuals) to identify genotyped but non-sequenced individuals who are likely undiagnosed cases of specific genetic diseases or likely pathogenic variant carriers. Notably, genotyping identifies particular genetic variants at predetermined locations in the genome (e.g., through one or more targeted assays), whereas sequencing determines the exact nucleotide sequence of a DNA segment (e.g., a DNA segment that is significantly longer than may be ascertained by a targeted assay for genotyping). Thus, sequencing can be used to identify pathogenic variants that would not be found in genotyping.

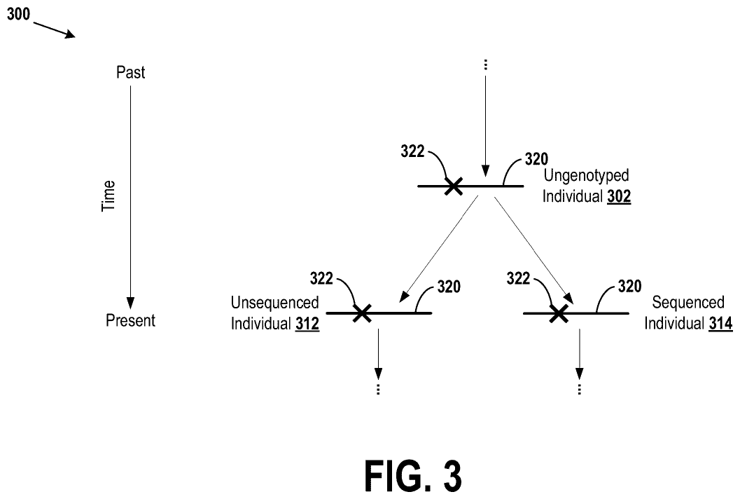

FIG. 3 depicts such a procedure 300. A pathogenic variant is present in an ancestor and also found in the same IBD segments within two descendants. For example, as illustrated in FIG. 3, an ungenotyped individual 302 may be an ancestor of an unsequenced individual 312 (a first descendant of the ungenotyped individual 302) and a sequenced individual 314 (a second descendant of the ungenotyped individual 302). The unsequenced individual 312 may have been genotyped for one or more conditions, but a full exome sequence may not have been determined for the unsequenced individual. Additionally, a full exome sequence may have been determined for the sequenced individual 314. Further, neither sequencing nor genotyping may have been performed for the ungenotyped individual 302. However, though sequencing and genotyping of the ancestor is not necessary to perform the techniques described herein, even in cases where sequencing and/or genotyping has been performed for the ancestor, the techniques described herein may still be performed.

As illustrated, an IBD segment 320 may be inherited from the ungenotyped individual 302 by the unsequenced individual 312 and the sequenced individual 314. The IBD segment 320 may include a pathogenic variant 322 (e.g., a disease-associated mutation). As such, both of the unsequenced individual 312 and the sequenced individual 314 may inherit the pathogenic variant 322. Additionally, the pathogenic variant 322 may be located at an ungenotyped site (e.g., a site of the ancestor 322 that has not been genotyped). Using the techniques described herein, upon identifying the pathogenic variant 322 within the whole exome sequence of the sequenced individual 314, the pathogenic variant 322 may be imputed to the unsequenced individual 312 (e.g., by identifying that the pathogenic variant 322 is included in the IBD segment 320 and that the IBD segment 320 is shared by the unsequenced individual 312). In some embodiments, the ungenotyped individual 302 may also be referred to as a “proband individual.”

When pathogenic variants are identified in an individual's genetic data, several actions can be taken depending on the clinical relevance and context of the findings. If the variant is associated with a known genetic disorder, healthcare providers may recommend further diagnostic testing, clinical evaluations, or genetic counseling to assess potential health risks and inheritance patterns. In cases where the variant is linked to a treatable or manageable condition, targeted interventions such as lifestyle modifications, regular monitoring, or preventative treatments may be implemented to reduce disease risk or delay onset. For certain inherited conditions, family members may also be tested to determine whether they carry the same pathogenic variant, allowing for early detection and proactive healthcare planning. Additionally, in the context of personalized medicine, the presence of specific pathogenic variants can guide treatment decisions, such as selecting targeted therapies that align with an individual's genetic profile. In some instances, online genomics web sites could provide a recommendation to the individual, such as “Based on your genetics we believe it is highly likely that you carry a disease-causing variant in gene XYZ. We recommend exome sequencing to confirm this and that you talk to a genetic counselor.”

In order to focus the genetic analysis on individuals most likely to benefit from it, the following factors may be taken into consideration. Any proband being considered should have a threshold level of genotyping in addition to phased whole exome sequencing. Pathogenic variants from certain chromosome regions may be excluded if not represented in a phasing panel being used. Recommendations might only be provided to an individual if that individual has a relative with shared IBD segments that are at least 5 centimorgans (CM). Phase of the pathogenic variant in relation to the statistically phased sequence (e.g., a phased version of genotyping information from a lab) can be used, but any phase conflicts are automatically dropped without attempting phase correction. Recommendations might only be made for likely pathogenic or pathogenic variants. Criteria for variant classification can vary but may involve the use of ClinVar (i.e., a freely accessible, public archive of reports of human variations classified for diseases and drug responses, with supporting evidence, that is provided by the National Center for Biotechnology Information (NCBI) at the National Library of Medicine (NLM) at the National Institute of Health (NIH)). This technique may be based on a specific list of genes, such as a list published by the American College of Medical Genetics and Genomics (ACMG) with or without modification (e.g., additions or removals of genes).

### IV. ALGORITHM

This section describes a family of related algorithms that can be used to detect the likelihood of a pathogenic variant in individuals. The source of probands was an exome sequencing dataset maintained for a plurality of individuals within a genetic services platform, but any whole exome or whole genome sequencing dataset could be used.

The classes of variants included those that were deemed pathogenic or likely to be pathogenic. The molecular consequences of these variants include loss of function of affected transcripts; i.e., nonsense mutations, splice site mutations, and insertion/deletion variants that result in downstream premature stop codons, or larger deletions removing either the first exon or more than 50% of the protein-coding sequence of the affected transcript. The molecular consequences of these variants also include missense mutations (genetic alterations in which a single base pair substitution alters the genetic code in a way that produces an amino acid that is different from the usual amino acid at that position, possibly altering the function of the resulting protein).

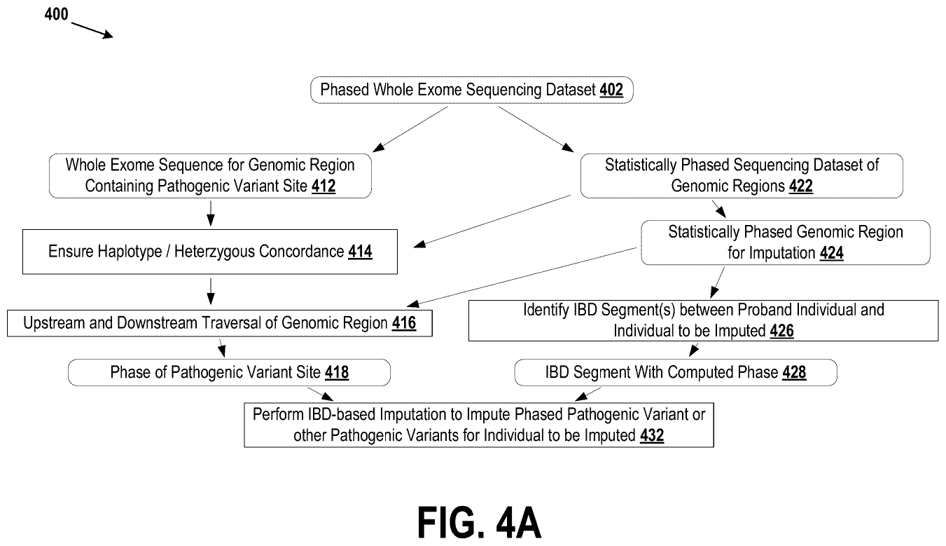

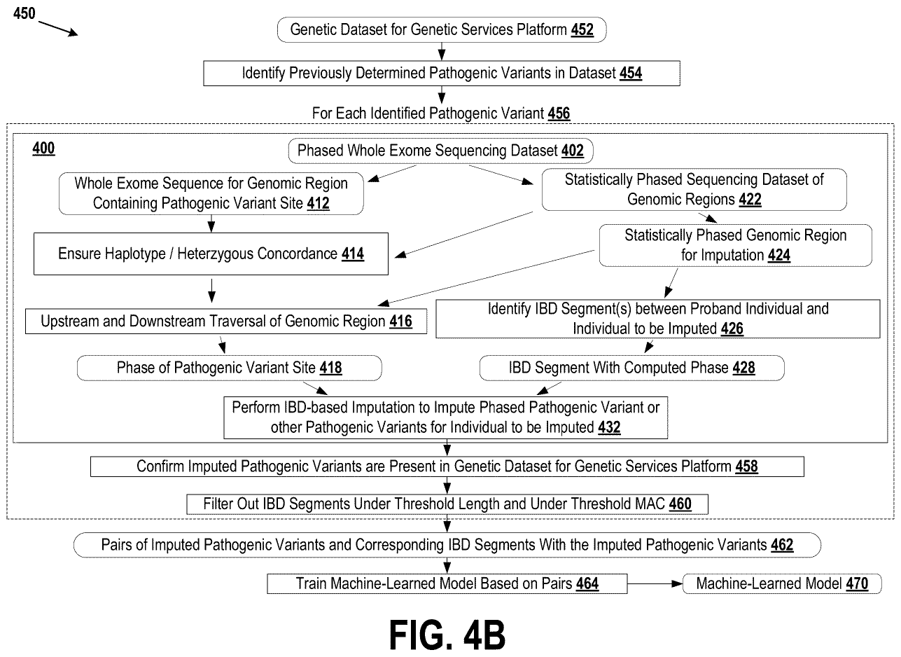

To validate this algorithm, variants with a minor allele count (MAC) between 2 and 10 (inclusive) from the whole exome sequencing dataset were selected. Here, minor alleles are those that are the second most common in a population of individuals. For each variant meeting this criterion, a proband was identified and an inference of the presence of the pathogenic allele in other probands for the same variant based solely on shared IBD segments was made. This process can be repeated for some or all variants and probands listed in the dataset. FIG. 4A provides an overview of the procedure 400.

As illustrated in FIG. 4A, the procedure 400 may begin by accessing a phased whole exome sequencing dataset 402. Upon retrieving the phased whole exome sequencing dataset 402, the procedure 400 may involve extracting a whole exome sequence for a genomic region containing a pathogenic variant site 412 from the phased whole exome sequencing dataset 402. The whole exome sequence for the genomic region that contains the pathogenic variant site may be a whole exome sequence corresponding to a proband individual, in some embodiments. Along with extracting the whole exome sequence for a genomic region containing a pathogenic variant site, the procedure 400 may include retrieving a statistically phased sequencing dataset of genomic regions 422. The statistically phased sequencing data may have been previously generated and stored for other purposes (e.g., within a genomic services platform) and/or determined based on one or more genotyping procedures performed for one or more individuals.

Next, the procedure 400 may include performing a haplotype and/or heterozygous concordance 414 determination. For example, the whole exome sequence for the genomic region containing the pathogenic variant site may be compared to a corresponding statistically phased sequence within the statistically phased sequencing dataset of genomic regions. The comparison may be performed to determine whether corresponding haplotypes and/or heterozygous pairs of alleles properly correspond within the dataset. In some embodiment, if there is inadequate correspondence, the whole exome sequence for the genomic region that contains the pathogenic variant site may be discarded and a different sequence may be retrieved for subsequent analysis.

The procedure 400 may also include retrieving a statistically phased genomic region for imputation 424 (e.g., retrieving the statistically phased genomic region for imputation from among genomic regions in the statistically phased sequencing dataset). In some embodiments, the genomic region may correspond to an individual for whom one or more genotyping procedures have been performed, but for whom whole exome sequencing has not been performed. As such, the procedure 400 may allow for the identification of phase and, consequently, for pathogenic variants within individuals for whom phase and pathogenic variants had not previously been identified. Further, the statistically phased genomic region may be the same region of the genome as the genomic region containing the pathogenic variant site for which the whole exome sequence was retrieved.

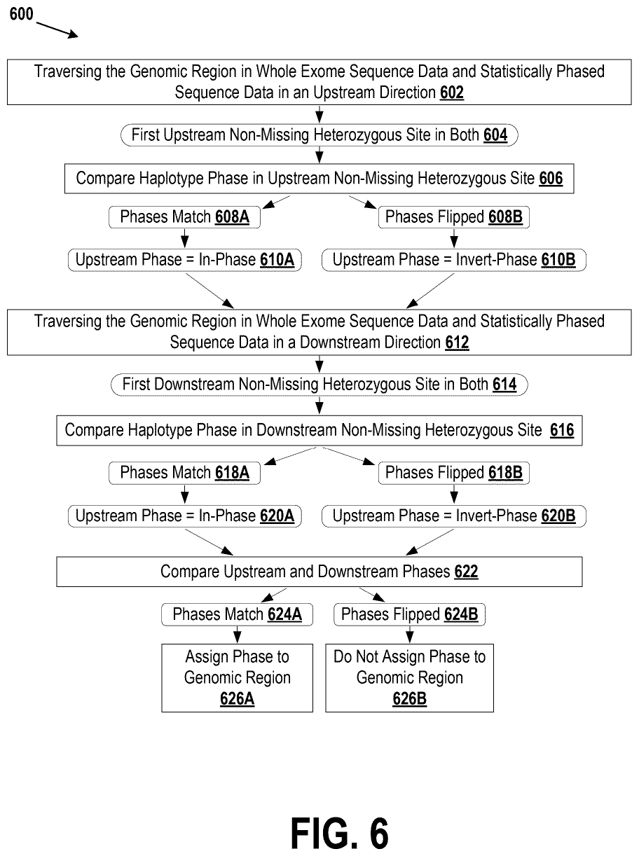

Subsequently, the procedure 400 may include performing an upstream and downstream traversal of the genomic region 416. The upstream and downstream traversal may be performed on the whole exome sequence for the genomic region and/or the statistically phased genomic region for imputation (e.g., which, itself, may include the pathogenic variant site). The upstream and downstream may be performed using the procedure 600 described below in further detail with reference to FIG. 6, for example. Additionally, the upstream and downstream traversal of the genomic region 416 may produce the phase of the pathogenic variant site 418.

In addition to determining the phase of the pathogenic variant site 418, the procedure 400 may include identifying one or more IBD segment(s) between a proband individual and an individual for which imputation is to be performed 426 (e.g., resulting in an IBD segment with computed phase 428). As noted above, the proband individual may be the individual for whom the whole exome sequence for the genomic region containing the pathogenic variant site was previously retrieved. However, in some embodiments, the proband individual may be a different individual (e.g., a different individual whose whole exome sequence data is contained within the whole exome sequencing dataset 402).

Further, the procedure 400 may include performing IBD imputation to impute the phased pathogenic variant or other pathogenic variants for the individual for which imputation is to be performed 432. As illustrated in FIG. 4A, the IBD-based imputation may be performed based on the phase of the pathogenic variant site 418 and the IBD segment with a computed phase 428.

A nearest-neighbor approach (detailed below) can be used to infer phase. Next, in-sample phased IBD within the whole exome sequencing dataset can be computed, to provide strict haplotype matching. This step may involve identifying shared IBD segments with a minimum length of 1.5 CM and covering at least 200 SNPs. Then, relatives who shared IBD within the pathogenic variant's genomic coordinates and matched the proband's haplotype can be identified. For these relatives, it was determined whether they also carried the pathogenic allele of interest.

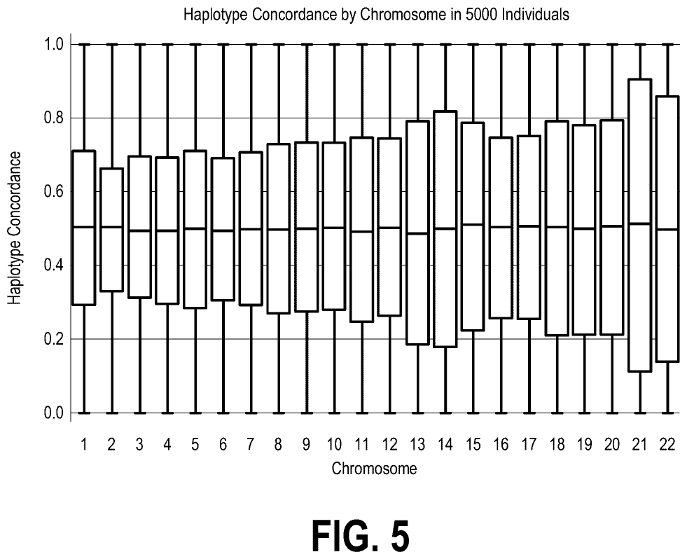

Inferring the presence of pathogenic variant carriers can also involve determining the phase of the pathogenic variant effect allele in relation to the statistically phased sequence data of the genomic region. While this information is typically retrievable, only a small subset of variants may be phased in the corresponding dataset. Further, it may be unlikely that rare variants are included in this subset. Additionally, phasing panels differ between the whole exome sequencing dataset and the statistically phased sequencing dataset. FIG. 5 shows the haplotype concordance for the same individual between the whole exome sequencing dataset and the statistically phased sequencing dataset (e.g., based on an example cohort of 5,000 individuals). Consequently, as shown in FIG. 5, the average haplotype concordance between genotyped phased data and sequencing-phased data within the same individuals is approximately 50% across various chromosomes.

Thus, one challenge present is how to infer the phase of the variant effect allele from exome sequencing in relation to the statistically phased sequencing data. A nearest neighbor phase inference approach may be used in some embodiments, as described below.

### A. Nearest Neighbor Phase Inference

Sequence data from a phased exome sequenced individual (could be phased with an external reference panel) is taken, the VCF/BCF container is converted to a whole exome sequence data entry, and for that same individual the statistically phased sequence data entry is identified. Neighboring heterozygous sites (since the pathogenic variant is typically ungenotyped and not present in the statistically phased sequence data entry) can be used to connect the phase of the whole exome-derived data entry with the statistically phased sequence data entry.

FIG. 6 depicts such a process 600, and is described algorithmically as follows.

1. Start the Upstream Walk-Begin walking upstream (relative to the pathogenic variant site) on both the whole exome sequence data and the statistically phased sequence data. Continue until encountering a non-missing heterozygous site that is present in both the whole exome sequence data and the statistically phased sequence data (e.g., where the whole exome sequence data and the statistically phased sequence data are both from the same individual). A non-missing heterozygous site may correspond to a site in the genome where there are two or more different alleles, and neither allele has a missing genotype.

2. Check Haplotype Matching-Compare the haplotypes of the upstream site between the whole exome sequence data and the statistically phased sequence data. If the phases of the haplotypes match, assign the upstream phase as in-phase (e.g., the assignment of alleles to each haplotype reflects their true, inherited grouping). If the phases do not match, assign the upstream phase as invert-phase (e.g., the phasing has been flipped or reversed relative to the true grouping).

3. Start the Downstream Walk-Repeat the process downstream, continuing until encountering a non-missing heterozygous site present in both the whole exome sequence data and the statistically phased sequence data (e.g., where the whole exome sequence data and the statistically phased sequence data are both from the same individual).

4. Assign Downstream Phase-Use the same procedure as described for the upstream walk. If the phases of the haplotypes match, assign the downstream phase as in-phase. If the phases do not match, assign the downstream phase as invert-phase.

5. Compare Upstream and Downstream Phases. If the upstream assigned phase matches the downstream assigned phase, return that phase. Otherwise, return none.

The steps of the process 600 described generally above are recited in more detail below with reference to FIG. 6. In some embodiments, the process 600 may correspond to the upstream and downstream traversal of the genomic region 416 shown and described above with reference to FIG. 4A. As such, the process 600 may be performed using a whole exome sequence for a genomic region that contains a pathogenic variant site 412 and a statistically phased genomic region for imputation 424.

The process 600 may begin by traversing the genomic region in the whole exome sequence data and the statistically phased sequence data in an upstream direction 602. In some embodiments, the whole exome sequence data and the statistically phased sequence data may correspond to the same individual (e.g., may be taken from the same genomic region of the same individual). Traversing the whole exome sequence data and the statistically phased sequence data may identify the first upstream non-missing heterozygous site in both the whole exome sequence data and the statistically phased sequence data 604.

Additionally, the process 600 may involve comparing the haplotype phase in the first upstream non-missing heterozygous site in the whole exome sequence data to the first upstream non-missing heterozygous site in the statistically phased sequence data 606. If the compared phases match 608A, the upstream phase may be assigned as in-phase 610A. If the compared phases are flipped 608B with respect to one another (or otherwise do not match), the downstream phase may be assigned as invert-phase 610B.

Further, the process 600 may involve traversing the genomic region in the whole exome sequence data and the statistically phased sequence data in a downstream direction 612. In some embodiments, the whole exome sequence data and the statistically phased sequence data may correspond to the same individual (e.g., may be taken from the same genomic region of the same individual). Traversing the whole exome sequence data and the statistically phased sequence data may identify the first downstream non-missing heterozygous site in both the whole exome sequence data and the statistically phased sequence data 614.

In addition, the process 600 may involve comparing the haplotype phase in the first downstream non-missing heterozygous site in the whole exome sequence data to the first downstream non-missing heterozygous site in the statistically phased sequence data 616. If the compared phases match 618A, the downstream phase may be assigned as in-phase 620A. If the compared phases are flipped 618B with respect to one another (or otherwise do not match), the downstream phase may be assigned as invert-phase 620B.

Still further, the process 600 may involve comparing the upstream phase to the downstream phase 622. If the phases match 624A (e.g., both the upstream phase and the downstream phase are in-phase or both the upstream phase and the downstream phase are invert-phase), the respective phase may be assigned to the genomic region that is represented in the whole exome sequence data and the statistically phased sequence data 626A. If the phases are flipped 624B (or otherwise do not match) (e.g., the upstream phase is in-phase and the downstream phase is invert-phase, or vice versa), no phase may be assigned to the genomic region that is represented in the whole exome sequence data and the statistically phased sequence data 626B.

As described above, FIG. 4A illustrates a procedure 400 usable to identify a proband and make an inference of the presence of a pathogenic allele in one or more other individuals (e.g., other probands) for the same variant based on shared IBD segment(s). According to example embodiments herein, the procedure 400 illustrated in FIG. 4A may also be augmented and/or modified in various ways. For example, in some embodiments, multiple IBD segments between a proband individual and an individual for imputation may be considered independently (e.g., at blocks 426, 428, and 432). Additionally or alternatively, MAC thresholds may be applied in order to exclude (or less favorably weight) alleles corresponding to pathogenic variants with lower MACs (e.g., as pathogenic variants with lower MACs may have less reliable phasing). For example, in some embodiments, pathogenic variants with an MAC below a threshold value (e.g., below 4, below 3, or below 2) may be excluded entirely (e.g., to eliminate those pathogenic variants that are most susceptible to sequencing and/or genotyping errors). In some embodiments (e.g., to account for the less reliable phasing of pathogenic variants with lower MACs), multiple phasing determinations may be performed using different genetic variants within a genomic region when the genetic variants have relatively low MACs (e.g., MACs between 1 and 10).

Further, in some embodiments, one or more quality metrics may be applied when considering proband IBD segments and/or when selecting a pathogenic variant from which to perform an upstream and downstream traversal. Such quality metrics may include a read depth associated with a sequencing protocol used to obtain an IBD segment and/or a pathogenic variant (e.g., a number of sequencing reads performed that support the presence of a pathogenic variant), an allelic balance associated with the sequencing protocol used to obtain an IBD segment and/or a pathogenic variant (e.g., a ratio describing the number of sequencing reads that contain the pathogenic variant at the pathogenic variant site relative to the number of sequencing reads that contain alternate alleles at the pathogenic variant site), and/or a genotyping quality (e.g., a confidence level) associated with a dataset from which data associated with an IBD segment and/or a pathogenic variant was obtained. Still further, in some embodiments, multiple IBD segments may be aggregated from different probands in order to compute an imputed pathogenic variant. For example, one recommendation for an individual (e.g., an indication of the presence of a pathogenic variant and/or a recommended treatment and/or prophylaxis based on the pathogenic variant) may be determined based on a first IBD segment and/or set of associated first quality metrics and another recommendation for the individual may be determined based on a second IBD segment and/or set of associated second quality metrics. These two recommendations may be combined or altered to generate a single notification to the individual based on the relative weights assigned to the two recommendations. In some embodiments, the IBD segments aggregated to make a recommendation may include IBD segment(s) that include the pathogenic variant and/or IBD segment(s) that exclude the pathogenic variant. In this way, both positive indicators of the presence of pathogenic variant alleles and negative indicators of the presence of pathogenic variant alleles may be weighed against one another to further refine the imputation.

In still other embodiments, one or more machine-learned models (e.g., similar to the machine-learned model 230 shown and described above with reference to FIGS. 2B and 2C) may be generated to assist in the imputation process. For example, FIG. 4B depicts a process 450 used to train a machine-learned model 470 usable to identify pathogenic alleles based on IBD segments. As illustrated in FIG. 4B, the process 450 may involve obtaining (e.g., retrieving) a genetic dataset for a genetic services platform 452. The dataset may include genetic sequences (e.g., a plurality of exome sequences, such as whole exome sequences) for a plurality of individuals. For example, the genetic dataset may be a dataset maintained for the purposes of genealogical study, genetic counseling, etc. The genetic dataset may include the genetic sequences for only those individuals who have provided consent for the individual's sequence data to be included in the genetic dataset or consent for the individual's sequence data to be used in scientific study.

Using the genetic dataset for the genetic services platform 452, the process 450 may then include identifying previously determined pathogenic variants in the dataset 454. In some embodiments, the dataset, itself, may include a list of pathogenic variants contained within the dataset (e.g., a list of all pathogenic variants in the dataset or lists of pathogenic variants associated with the various sequences and/or individuals in the dataset). The list(s) may have been generated as part of previous genetic risk assessment(s) for the individual(s), for example. Further, identifying previously determined pathogenic variants may involve selecting a subset (e.g., all) of the pathogenic variants from the list(s) (e.g., according to one or more predetermined criteria). Alternatively, in some embodiments, identifying previously determined pathogenic variants 454 may involve cross-referencing a list of known pathogenic variants with the particular exome sequence(s) included in the genetic dataset 452.

Then, for each identified pathogenic variant, the process 450 may include a subprocess 456. The subprocess 456 may include imputing a phased pathogenic variant or other pathogenic variants for an individual based on the identified pathogenic variant. This may involve performing the procedure 400 described above with respect to FIG. 4A using the whole exome sequence for a genomic region containing the pathogenic variant site 412 that corresponds to the particular identified pathogenic variant being considered in a given instance of the subprocess 456. For example, the whole exome sequence for a genomic region containing the pathogenic variant site 412 may be retrieved from the phased whole exome sequencing dataset 402 based on the identified pathogenic variant being considered in the given instance of the subprocess 456. The steps of the subprocess 456 then may proceed based on the whole exome sequence for the genomic region containing the pathogenic variant site 412 and the corresponding statistically phased sequencing dataset of genomic regions 422 (e.g., as described in detail above with respect to FIG. 4A).

Upon imputing the phased pathogenic variant or other pathogenic variants for the individual to be imputed 432 in a given instance of the subprocess 456, step 458 may be performed. Step 458 may involve confirming that the imputed pathogenic variants are present in the genetic dataset for the genetic services platform 452 (e.g., to confirm that the performed imputation actually resulted in an accurate pathogenic variant). If the imputed pathogenic variant(s) are not in the genetic dataset for the genetic services platform 452, the imputed genetic variant(s) and/or the associated IBD segments (e.g., determined at steps 426 and 428) may be discarded and the process 450 may proceed to the next instance of the subprocess 456 (e.g., using a different previously determined pathogenic variant). Next, the subprocess 456 may proceed to step 460. At step 460, identified IBD segments (e.g., paired with the imputed phase of the pathogenic variant or other imputed pathogenic variant(s)) that do not exceed a threshold length (e.g., exceed 5 cM) and/or do not exceed a threshold MAC value (e.g., 1) may be discarded. The process 450 may then proceed to the next instance of the subprocess 456. By performing step 460, the robustness of the machine-learned model 470 trained according to the process 450 may be further enhanced. Upon completion of the subprocess 456 for each of the identified pathogenic variants, the process may result in a set of pairs of: (i) imputed pathogenic variants (e.g., imputed at step 432) and (ii) the correspondingly identified IBD segments (e.g., identified at steps 426 and 428).

Step 464 may be performed using these pairs of imputed pathogenic variants and the correspondingly identified IBD segments. In some embodiments, step 464 may also be performed based on the original phased exome sequence data from which the imputed pathogenic variant was imputed (e.g., the whole exome sequence for the genomic region containing the pathogenic variant site 412 from the corresponding instance of subprocess 456). At step 464, the process 450 may include training a machine-learned model 470 based on the pairs. Training the machine-learned model 470 may be performed according to the process shown and described above with reference to FIG. 2B (e.g., where the pairs of imputed pathogenic variants and the correspondingly identified IBD segments and/or the whole exome sequence for the genomic region containing the pathogenic variant site 412 from the corresponding instance of subprocess 456 are used as the training data 290). In some embodiments, the machine-learning training algorithm 220 used in step 464 of the process 450 may include the use of logistic regression, a random forest, and/or eXtreme Gradient Boosting (XGBoost), among other possibilities. Once the machine-learned model 470 is trained, the machine-learned model 470 may be used in future cases to generate a prediction (e.g., as in the process shown and described above with reference to FIG. 2C). For example, an imputed phase for a pathogenic variant or other pathogenic variants may be determined using the machine-learned model 470 based on a whole exome sequence for a genomic region of an individual that contains a pathogenic variant site. Alternatively, an imputed phase for a pathogenic variant or other pathogenic variants may be determined using the machine-learned model 470 based on one or more IBD segments (e.g., from one or more proband individuals) identified for an individual. In this way, the machine-learned model 470 may further expedite the process of imputing phase for a pathogenic variant or other pathogenic variants for an individual (e.g., thereby further conserving computational resources, such as memory, processing power, and communication bandwidth).

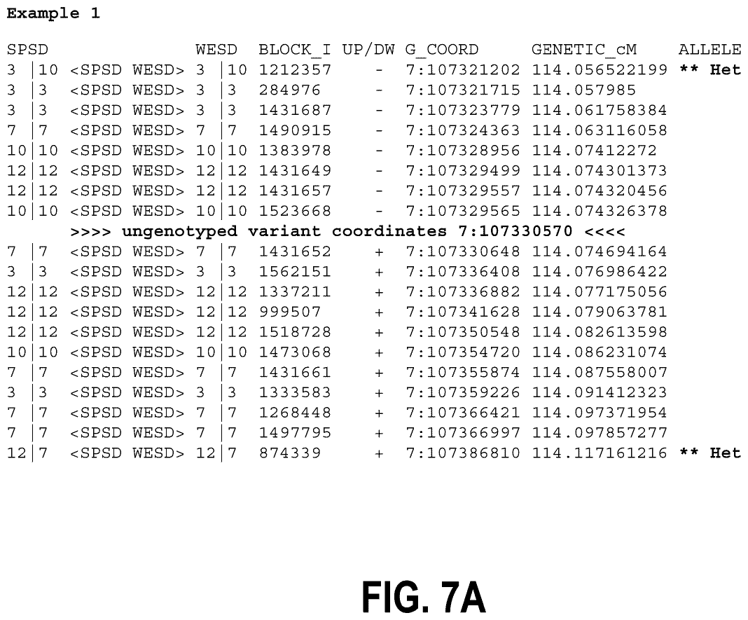

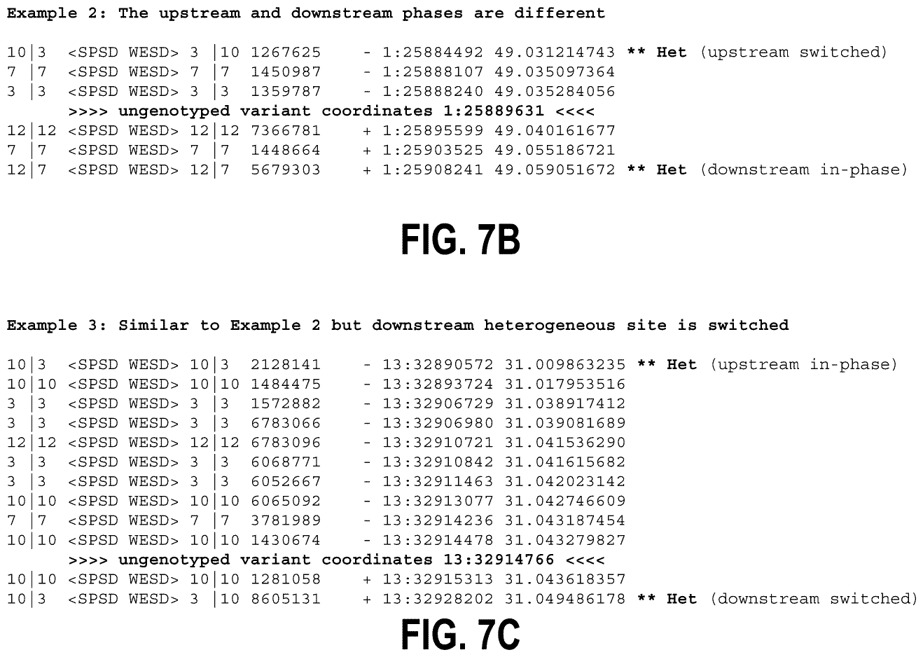

FIGS. 7A, 7B, and 7C provide examples of the operation of this algorithm. In FIGS. 7A-7C, “SPSD” represents “statistically phased sequence data,” “WESD” represents “whole exome sequence data,” “UP/DW” represents “upstream/downstream” (where “−” corresponds to upstream and “+” corresponds to downstream), “G_COORD” represents the genomic coordinate, “GENETIC_cM” represents the genetic location measured in cM, and “ALLELE” indicates the zygosity of the respective allele (where no ALLELE value corresponds to homozygous and ** Het corresponds to heterozygous). In Example 1, of FIG. 7A, from the pathogenic variant coordinates at 7:107330570, the walk proceeds upstream until a heterozygous site is reached (in this case at 7:107321202), and the alleles and phase match is checked. Since they match, the upstream heterozygous site is recorded as in-phase because they are both in the same phase. Similarly, the walk proceeds downstream until a heterozygous site is reached at 7:107386810, and the same check is performed. This downstream heterozygous site is also in-phase. This means the haplotypes from the statistically phased sequence data can be directly used. In Example 2 of FIG. 7B, the phase in relation to the statistically phased sequence cannot be reliably inferred (e.g., because the upstream heterozygous site is invert-phase while the downstream heterozygous site is in-phase), so this portion of the statistically phased sequence is dropped from the analysis. Similarly, in Example 3 of FIG. 7C, the phase in relation to the statistically phased sequence cannot be reliably inferred (e.g., because the upstream heterozygous site is in-phase while the downstream heterozygous site is invert-phase), so portion of the statistically phased sequence is dropped from the analysis.

The X chromosome contains two pseudo-autosomal regions (PARs); short regions of homology between the X and Y chromosomes. In the database used herein, approximately 23% heterozygosity is observed within the X-PAR region. However, when X-PAR regions are not represented in a phasing dataset they can be excluded from analyses.

The techniques herein can include variants selected manually, from loss of function/missense variants, or some combination thereof. Also, internal variant selection criteria can be used.

### B. HybridIBD

HybridIBD is an IBD detection algorithm that combines strengths from two separate IBD algorithms to improve IBD detection accuracy. The first algorithm is phasedIBD, which compares matches between phased genomes separated into distinct parental contributions. It excels at finding the short IBD segments that connect individuals to distant relatives. With phasedIBD, segments down to 5 cM or shorter can be identified reliably. The second algorithm is IBD64, which works by comparing unphased genomes. This gives it an advantage for finding long IBD segments shared between close relatives since these are often considerably fragmented by phasing errors. With HybridIBD, every new genome is first analyzed by phasedIBD to find distant relatives. Then, any pair sharing a significant amount of IBD has refined their IBD using IBD64. This dual approach leverages the strengths of each algorithm, improving the overall accuracy.

### C. Proband Selection

Variants shortlisted from ClinVar based on the variant selection filtering criteria above can be used to identify individuals in the reference exome from the whole exome sequencing dataset who carry the pathogenic allele of each selected variant. These individuals are identified as probands and serve as key reference points for further analysis. For each proband, HybridIBD is computed against all the genotyped individuals in a reference database. This process involves finding shared stretches of DNA segments between the proband and the rest of the individuals in the database. This enables identifying of DNA segments that have been inherited from a common ancestor—which can be used for determining shared pathogenic alleles. Particularly, phasedIBD parameters were tuned to adjust sensitivity to mismatches, and the templated positional Burrows-Wheeler transform (TPBWT) templates were set to [[1,1,0], [1,0,1], [0,1,1]] which guarantees all matches will be found as long as no more than one mismatches is in any three SNP-window. Further, IBD segments as short as 1.5 cM that contain at least 200 SNPs (with the default being 3.0 cM and 300 SNPs) to be considered were allowed. Additionally, IBD segments in known high linkage disequilibrium (LD) regions were filtered out.

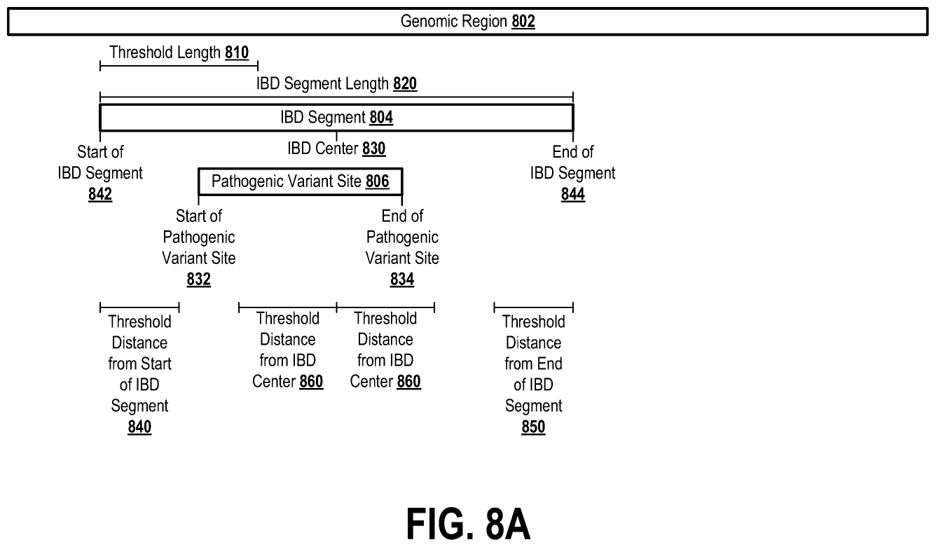

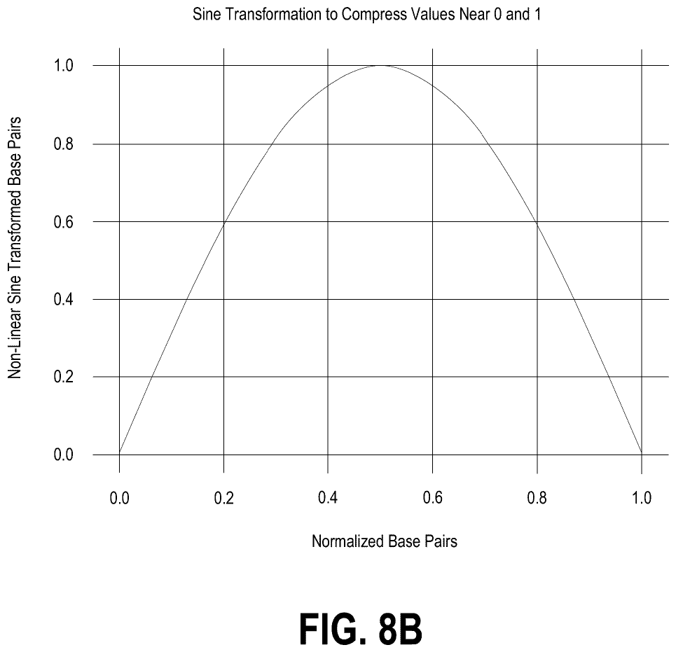

Other selection criteria may be applied to select an appropriate IBD segment, appropriate genomic region containing an IBD segment, or an appropriate proband individual having an IBD segment and/or genomic region. FIG. 8A illustrates various selection criteria that may be applied to the selection. One or more (e.g., all) of the selection criteria illustrated in FIG. 8A may be implemented jointly. As illustrated, a candidate IBD segment 804 that includes the pathogenic variant site 806 may be considered. The candidate IBD segment 804 may represent a segment of a genomic region 802, for example.

As illustrated in FIG. 8A, one selection criterion may include a start 832 or an end 834 of the pathogenic variant site 806 exceeding a threshold distance 840 from a start 842 of the IBD segment 804. Additionally or alternatively, one selection criterion may include a start 832 or an end 834 of the pathogenic variant site 806 exceeding a threshold distance 850 from an end 844 of the IBD segment 804. Further, one selection criterion may include a start 832 or an end 834 of the pathogenic variant site 806 being within a threshold distance 860 of a center 830 of the IBD segment 804. For example, as illustrated in FIG. 8A, the end 834 of the pathogenic variant site 806 may be within the threshold distance 860 of the center 830 of the IBD segment. In addition, as illustrated in FIG. 8A, one selection criterion may include a length 820 of the IBD segment 804 exceeding a threshold length 810 (e.g., a threshold length of 1.5 cM or 200 SNPs). By applying one or more selection criteria when choosing a candidate IBD segment and/or a candidate proband individual, the number of false positives (e.g., the number of imputed shared pathogenic variant sites determined based on the IBD segment 804) may be reduced.

### D. Recommendation Flow

To generate the list of potential recommendations, each proband and variant combination may use: (1) variant information such as the inferred phase of the variant minor allele from whole exome sequencing dataset using the nearest neighbor phase inference method, and (2) HybridIBD results for that proband compared to all the qualifying individuals in a reference database, containing haplotype of the IBD segment matches along with genomic coordinates of the IBD segment start and end. With this information, all the relatives that are IBD at the variant genomic coordinates can be iterated over and their genotype id, shared genomic coordinates, and shared segment length can be stored. For each relative, a posterior probability of confidence that the relative and the proband share a variant pathogenic allele can be computed.

### E. Posterior Probability of Recommendation

In the context of IBD recommendations, the probability of a relative carrying the pathogenic variant allele can be predicted based on features such as shared IBD segment length and the relative position of the variant within the shared IBD segment. Posterior probability can be expressed as:

$$P ( Y = 1 \vert X ) = \frac{P ( X \vert Y = 1 ) P ( Y = 1 )}{P ( X )}$$

Posterior probability P(Y=1|X) is the probability of receiving a true health recommendation Y=1 after considering the IBD sharing features X. Prior Probability P(Y=1) is an inference that, based on historical data, a person has, for example, a 10% chance of receiving a true health recommendation. Likelihood P(X|Y=1) represents how likely it is to observe the features (X, such as IBD shared segment length) given that the person receives a true recommendation Y=1. This is learned/estimated by the classification model from the data. Evidence P(X) is the total probability of observing the IBD segment features, considering all possible classes (both receiving and not receiving the recommendation) to normalize the posterior probability.

A classification model, such as a logistic regression model, can be fit to estimate the posterior probabilities for a recommendation. The model estimates the coefficients β₀, β₁, . . . , βn for a plurality of multi-dimensional features (e.g., IBD sharing segment length, sine transformed base pair location, sequencing quality metrics, etc.). The model then computes the probability of receiving a health recommendation Y=₁ using the sigmoid function:

$$P ( Y = 1 \vert X ) = \frac{1}{1 + {e}^{- ( {β}_{0} + {β}_{1} {X}_{1} + + {β}_{2} {X}_{2} + + {β}_{n} {X}_{n} )}}$$

Where X₁, X₂, . . . , Xₙ are the features and β₀, β₁, . . . , βₙ are the coefficients learned by the model during training. The sigmoid function maps the unbounded linear combination of features to a standardized output range between 0 and 1, representing a calibrated probability.

The features in the model fit are derived from the hybrid IBD results, proband sequencing metrics, and inferred phase metrics, as detailed below. For each proband-relative pair the following can be derived: shared IBD segment length and sine-transformed position of the variant. The shared IBD segment length may represent the length of an IBD segment shared between two individuals (measured in cM). The sine-transformed position of the variant may be calculated in the following manner:

Using the base pair position of a genetic variant (e.g., the start and the end of the shared IBD segment), a linear relative transformation is determined, such as

$${R}_{linear} ( v ) = \frac{v - {v}_{min}}{{v}_{max} - {v}_{min}}$$

Where: v is the target genomic base pair coordinate with a given range of IBD segment start and end, vₘᵢₙ is the IBD segment start genomic coordinate, vₘₐₓ is the IBD segment end genomic coordinate, and Rₗᵢₙₑₐᵣ(v) is the normalized position as a fraction of the genomic range of the IBD segment. A sine transformation may then be applied such that Rₙₒₙₗᵢₙₑₐᵣ(v) represents a variable that emphasizes central values while compressing values near boundaries, such as:

$${R}_{nonlinear} ( v ) = sin ( π \cdot {R}_{linear} ( v ) )$$

FIG. 8B depicts an example of the sine-transformed position of the variant given a start and end of IBD segments. IBD segments can be prioritized based on pathogenic variant location (e.g., with IBD segments having a pathogenic variant located nearer to the center of the IBD segment, as determined based on the sine transformation, having higher priority). Segments with variants closer to the midpoint may be given higher priority, while segments with variants at the start or end may be given lower priority.

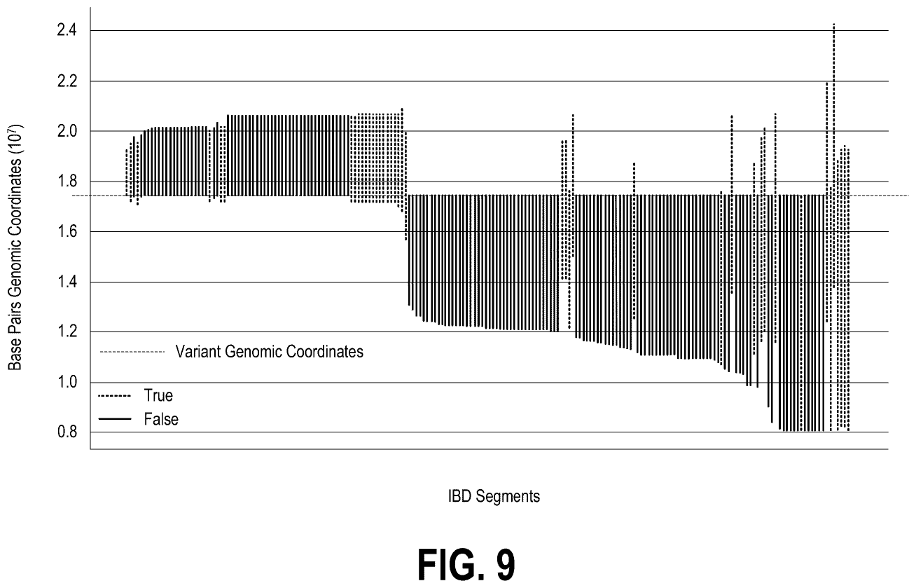

FIG. 9 illustrates false positives for IBD segments as determined based on the location of a pathogenic variant within the IBD segment. The x-axis represents various IBD segments, and the y-axis represents base pair genomic coordinates. The genomic coordinate of the variant is marked by a horizontal dashed line. Dashed vertical lines indicate true positives, and solid vertical lines indicate false positives. Notably, the variant's genomic coordinate (horizontal dashed line) is only present at the very edge of the respective IBD segment for the vast majority of the false positives (solid vertical lines), illustrating the value of using a sine-transformed positional feature to differentiate the false positives from the true positives.

X chromosome variants can be represented as a Boolean feature indicating whether the variant is on the X chromosome. This may assist in identifying and/or addressing phase inference failures that occur predominantly on the X chromosome.

Larger shared IBD segments may indicate closer genetic ties, which could increase the probability of shared genetic variants relevant to the recommendation. Similarly, variants near the center of an IBD segment are more likely to be true IBD-inherited than those near the edges (e.g., as illustrated in FIG. 8B), which could be due to partial phasing errors or other technical artifacts.

The methods described herein may include a “pile-up” architecture that simultaneously aggregates IBD segments from a positive proband set (carrying the variant pathogenic allele) and a negative proband set (wild-type individuals not having variant pathogenic allele) to differentiate true pathogenic signals from background noise. To reduce sequencing artifacts, example methods may include performing a preliminary maximization step that prioritizes the longest shared IBD segments and the highest-confidence sequencing metrics (Genotype Quality (GQ) score, Variant Allele Frequency (VAF), Phred-scaled Likelihood (PL)) for each variant-proband combination before final aggregation. Further, example embodiments may convert disparate segment data into a stable, higher-level representation by calculating centrality measures (mean, median, max/min) across multiple independent IBD matches, effectively “smoothing” individual data fluctuations into a consistent evidence profile to a total of 33 features, including post-aggregation summary statistics.

The non-aggregated features are described below.

### 1. Ibd Features:

Length (cM)
Sine-transformed base pairs (bp)
   2. Variant-Level Features—these Features Describe Characteristics of Individual Genetic Variants:
Chromosome X (chrom_x): A boolean indicator for variants located on chromosome X, which may follow distinct inheritance patterns compared to autosomes.
Minor Allele Count (mac): The count of the less-frequent allele in the population, serving as a measure of allele frequency. Rare variants may offer more information, but may also be less reliably detected.
Insertion/Deletion (is_indel): Indicates whether the variant is an insertion or a deletion.
Insertion/Deletion Flags (is_insertion/is_deletion): Specific flags to differentiate between insertion and deletion events.
Indel Length: The absolute size of the insertion or deletion, providing information on how indel size might correlate with quality or reliability.

### 3. Phasing Features:

Proportion of Missing Phase Calls at Variant (phased_prop_at_variant): This metric reflects the proportion of missing phase calls at a genomic location using the nearest-neighbor phasing method. Higher values suggest greater uncertainty in the phasing process.
   4. Sequencing Features—these Features Relate to the Quality and Characteristics of Sequencing Data:
Genotype Quality Score (GQ): A statistical confidence score for the genotype call.
Depth of Sequencing Reads (DP_prise): The number of sequencing reads supporting the variant, indicating the coverage depth.
Variant Allele Frequency (VAF): The proportion of reads carrying the alternate allele, used to validate if the observed ratio is consistent with the genotype.
Allelic Depths (AD1/AD2): The depths of sequencing reads for the reference and alternate alleles, respectively.
Phred-scaled Likelihoods (PL0/PL1/PL2): Probabilistic scores (Phred-scaled) for the three possible genotypes (0/0, 0/1, 1/1), which may offer a more nuanced view of genotype uncertainty beyond a single call.

### V. MODEL PERFORMANCE

Three main machine learning models were trained to evaluate how well shared IBD segment features and sequencing metrics predict the posterior probability of a given IBD-based recommendation. The models investigate the likelihood that an individual should receive a recommendation based on the aggregated characteristics of their shared DNA segments. The architectures evaluated include Logistic Regression (as a linear baseline), Random Forest (as a non-linear bagging model), and XGBoost (utilizing gradient boosting with regularization).

The dataset used for validation analyses included multiple genetic variants. For every proband-relative pair, the dataset incorporates ground-truth genotype information obtained from whole exome sequencing. This means that, for each aggregated IBD segment shared between a proband and their relative, there are detailed multi-dimensional features (e.g., segment length, sine-transformed base-pair position, and read depths) and a binary outcome indicating whether the proband and relative share the pathogenic variant allele.

To optimize the models and balance overfitting, generalization, and probability calibration (especially at high-confidence thresholds), a data-splitting strategy was employed. All model training was performed on a designated training set, while hyperparameter tuning was iteratively evaluated on a separate validation set. A final test set was strictly reserved and kept blinded until after the model architectures were finalized to ensure unbiased, real-world inference.

The methodology described herein uses the aggregated, high-dimensional feature space. Random Forest and XGBoost were specifically integrated to capture the complex, non-linear relationships between variant-level sequencing metrics and IBD segment characteristics.

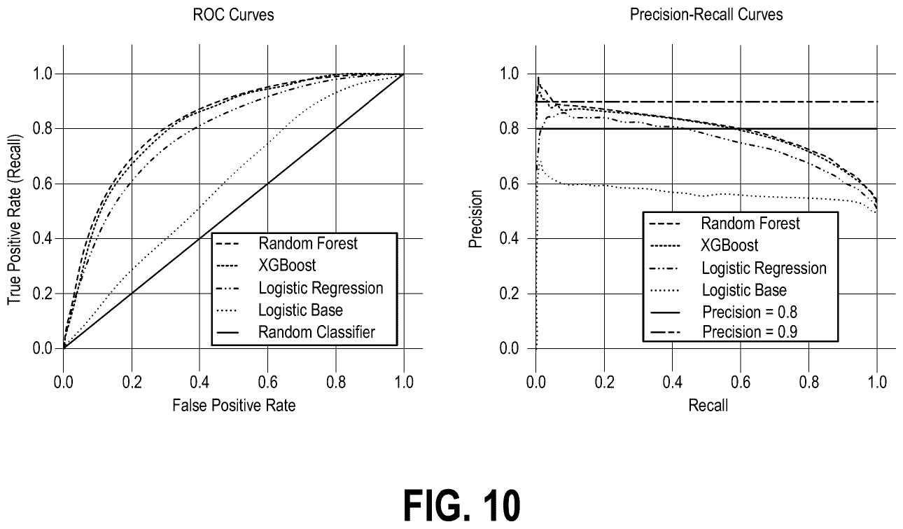

The results of these updated models, as measured in the validation cohort, are illustrated in FIG. 10.
As illustrated, Random Forest achieved the highest overall area under the curve (AUC=0.821) and the strongest balance of precision (0.744) and recall (0.754) at a 0.5 probability threshold. XGBoost performed similarly, yielding a slightly higher recall (0.765) with a marginal trade-off in precision. Further, at stricter probability thresholds (0.8-0.9), both Random Forest and XGBoost maintained exceptionally high precision (>0.85), confirming the suitability and reliability for high-confidence applications of both Random Forest and XGBoost (performance on validation set is illustrated in the table below).

Model Performance Metrics in the validation cohort
at various posterior probability thresholds

| | | Precision | Precision | Precision | Recall | Recall | Recall |
|---|---|---|---|---|---|---|---|
| Model | AUC | @ 0.5 | @ 0.8 | @ 0.9 | @ 0.5 | @ 0.8 | @ 0.9 |
| XGBoost | 0.818 | 0.732 | 0.846 | 0.87 | 0.765 | 0.348 | 0.077 |
| Logistic Regression | 0.782 | 0.712 | 0.839 | 0.854 | 0.714 | 0.193 | 0.061 |
| Random Forest | 0.821 | 0.744 | 0.86 | 0.89 | 0.754 | 0.266 | 0.05 |

### VI. EVALUATION

To ensure robust generalizability and prevent overfitting, the system's predictive architectures were evaluated against an independent, strict hold-out dataset. None of these variants were exposed to the models during the training or hyperparameter tuning phases. The evaluation focused on generating posterior probabilities for this independent cohort, specifically isolating a validation subset of recommendations corresponding to actual individuals (n=22, spanning a MAC range of 2 to 325). To establish ground truth, a concordance verification step was executed that included confirming that the variant imputed by the current methodology was physically present in the corresponding individual's VCF file, thereby validating the imputation against direct sequencing data.

Precision, Recall, and F1 scores of the model's performance
as evaluated in an independent test set. This evaluation
included 22 individuals with clinical whole exome sequencing
data, with MAC ranges from 2 to 325

| Model | Threshold | Precision | Recall | F1 |
|---|---|---|---|---|
| XGBoost | 0.5 | 0.875 | 0.778 | 0.824 |
| Logistic Regression | 0.5 | 0.867 | 0.722 | 0.788 |
| Random Forest | 0.5 | 0.889 | 0.889 | 0.889 |

Genetic imputation is a statistical technique used to estimate an individual's genotype at ungenotyped genomic locations by leveraging information from typically a large reference panel of sequenced individuals. The method assigns a probability for each possible genotype-homozygous rare, heterozygous, or homozygous common—based on observed patterns of LD. By comparing an individual's genotyped variants to haplotypes in the reference panel, imputation algorithms can predict missing genotypes with high confidence, filling in gaps where direct genotyping data is unavailable. This approach is widely used in genome-wide association studies (GWAS).

Unlike traditional imputation (e.g., R7 imputation), the IBD-based imputation approach described herein does not rely on reference panels or LD patterns. Instead, this technique infers the presence of a variant allele conditional that an individual shares a relatively long IBD segment (>=5 cM) with a known carrier (e.g., proband from truth sequencing data in the whole exome sequencing dataset). The method assumes that if two individuals share an IBD segment, there is a high likelihood that they may also share genetic variants within that segment. Some of the key differences between the two techniques are summarized below.

| | Traditional Genetic Imputation | IBD-Based Imputation |
|---|---|---|
| Source of | Reference panel haplotypes | Direct Next Generation Sequencing |
| Information | (population-based) | (family/population) |
| Resolution | High for common variants, lower | Effective for rare variants shared |
| | for rare ones | within families/distant relatives |
| Applicability | GWAS, polygenic risk scores | Carrier inference, rare variant |
| | | discovery |
| Assumptions | LD structure is well-defined in | IBD segments carry the same variant |
| | population | between individuals |
| Limitations | Poor precision with rare variants | Performance is high across lower |
| | not well represented in reference | Minor Allele Counts (2-10), Recall is |
| | panels but high recall. | significantly low. |
| | Ancestry dependent because of | Ancestry agnostic. |
| | reliance on LD patterns. | |

### VII. EXAMPLE OPERATIONS

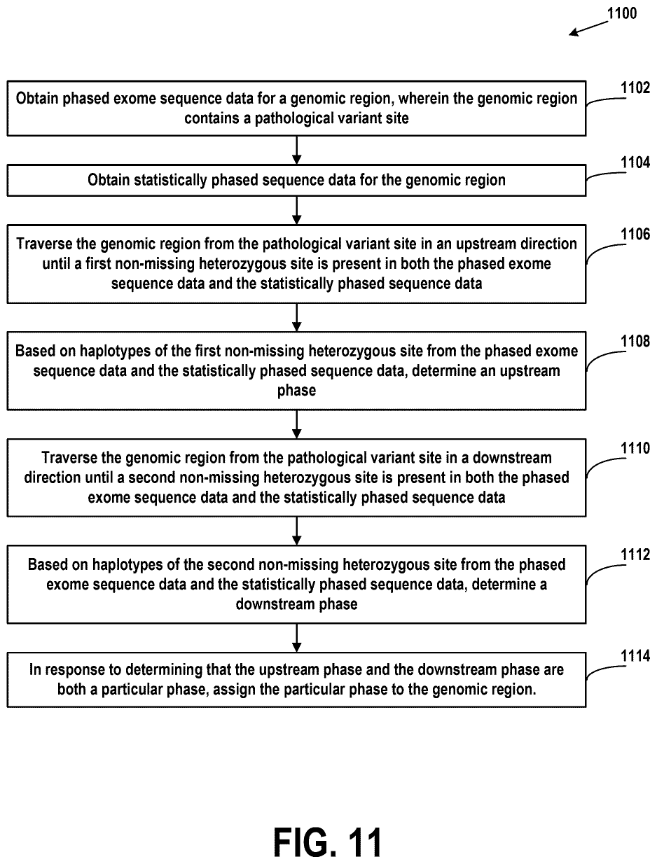

FIG. 11 is a flow chart illustrating an example embodiment. The process 1100 illustrated by FIG. 11 may be carried out by a computing device, such as computing device 100, and/or a cluster of computing devices, such as server cluster 200. However, the process can be carried out by other types of devices or device subsystems. The embodiments of FIG. 11 may be simplified by the removal of any one or more of the features shown therein. Further, these embodiments may be combined with features, aspects, and/or implementations of any of the previous figures or otherwise described herein.

Block 1102 may involve obtaining phased exome sequence data for a genomic region, wherein the genomic region contains a pathogenic variant site.

Block 1104 may involve obtaining statistically phased sequence data for the genomic region.

Block 1106 may involve traversing the genomic region from the pathogenic variant site in an upstream direction until a first non-missing heterozygous site is present in both the phased exome sequence data and the statistically phased sequence data.

Block 1108 may involve, based on haplotypes of the first non-missing heterozygous site from the phased exome sequence data and the statistically phased sequence data, determining an upstream phase.

Block 1110 may involve traversing the genomic region from the pathogenic variant site in a downstream direction until a second non-missing heterozygous site is present in both the phased exome sequence data and the statistically phased sequence data.

Block 1112 may involve, based on haplotypes of the second non-missing heterozygous site from the phased exome sequence data and the statistically phased sequence data, determining a downstream phase.

Block 1114 may involve, in response to determining that the upstream phase and the downstream phase are both a particular phase, assigning the particular phase to the genomic region.

Some embodiments of the process 1100 may further involve: determining a proband individual with an identity-by-descent (IBD) segment matching the particular phase and within the genomic region; determining a further individual that shares the IBD segment; and generating a notification the further individual regarding the pathogenic variant site.

In some embodiments of the process 1100, determining the proband individual comprises determining that the IBD segment has minimum length of 1.5 centimorgans and includes at least 200 single nucleotide polymorphisms.

Some embodiments of the process 1100 may further involve determining that a start or an end of the pathogenic variant site exceeds a threshold distance from a start of the IBD segment.

Some embodiments of the process 1100 may further involve determining that a start or an end of the pathogenic variant site exceeds a threshold distance from an end of the IBD segment.

Some embodiments of the process 1100 may further involve determining that a start or an end of the pathogenic variant site is within a threshold distance of a center of the IBD segment.

Some embodiments of the process 1100 may further involve determining that a length of the IBD segment exceeds a threshold length.

Some embodiments of the process 1100 may further involve applying a targeted therapy to the further individual, and wherein the targeted therapy is based on a pathogenic variant associated with the pathogenic variant site.

In some embodiments of the process 1100, the targeted therapy may involve a preventative treatment that reduces a disease risk or delays a disease onset associated with the pathogenic variant. For example, the targeted therapy may involve a preventative treatment that reduces a disease risk or delays a disease onset for a disease identified by the ACMG based on pathogenic or likely pathogenic variants (e.g., in the ACMG secondary findings (SF) v3.2 list, such as familial adenomatous polyposis (FAP); familial medullary thyroid cancer; multiple endocrine neoplasia 2; hereditary breast cancer, hereditary ovarian cancer; hereditary paraganglioma-pheochromocytoma syndrome; juvenile polyposis syndrome (JPS); hereditary hemorrhagic telangiectasia syndrome; Li-Fraumeni syndrome; Lynch syndrome (hereditary nonpolyposis colorectal cancer (HNPCC)); multiple endocrine neoplasia type 1; MUTYH-associated polyposis (MAP); NF2-related schwannomatosis; Peutz-Jeghers syndrome (PJS); PTEN hamartoma tumor syndrome; retinoblastoma; tuberous sclerosis complex; von Hippel-Lindau syndrome; WT1-related Wilms tumor; aortopathies; arrhythmogenic right ventricular cardiomyopathy; catecholaminergic polymorphic ventricular tachycardia; dilated cardiomyopathy; Ehlers-Danlos syndrome, vascular type; familial hypercholesterolemia; hypertrophic cardiomyopathy; long QT syndrome types 1 and 2; long QT syndrome 3; Brugada syndrome; long QT syndrome types 14-16; biotinidase deficiency; Fabry disease; ornithine transcarbamylase deficiency; Pompe disease; hereditary hemochromatosis; hereditary hemorrhagic telangiectasia; malignant hyperthermia; maturity-onset of diabetes of the young; RPE65-related retinopathy; Wilson disease; or hereditary TTR (transthyretin) amyloidosis).

Some embodiments of the process 1100 may further involve: determining a proband individual with an identity-by-descent (IBD) segment matching the particular phase and within the genomic region; determining a further individual that shares the IBD segment; identifying a secondary pathogenic variant site within the IBD segment; and generating a notification to the further individual regarding the secondary pathogenic variant site.

Some embodiments of the process 1100 may further involve: determining a first proband individual with a first identity-by-descent (IBD) segment matching the particular phase and within the genomic region; determining a second proband individual with a second IBD segment matching the particular phase and within the genomic region; determining a further individual that shares the first IBD segment and the second IBD segment; selecting, based on a sine-transformed position of the pathogenic variant site within the first IBD segment and a sine-transformed position of the pathogenic variant site within the second IBD segment, the first IBD segment; and generating a notification to the further individual regarding the pathogenic variant site based on the first proband individual or the first IBD segment.

In some embodiments of the process 1100, the upstream phase may be in-phase when the haplotypes of the first non-missing heterozygous site from the phased exome sequence data and the statistically phased sequence data match. Similarly, the upstream phase may be invert-phase when the haplotypes of the first non-missing heterozygous site from the phased exome sequence data and the statistically phased sequence data do not match.

In some embodiments of the process 1100, the downstream phase may be in-phase when the haplotypes of the second non-missing heterozygous site from the phased exome sequence data and the statistically phased sequence data match. Similarly, the downstream phase may be invert-phase when the haplotypes of the second non-missing heterozygous site from the phased exome sequence data and the statistically phased sequence data do not match.

In some embodiments of the process 1100, a minor allele count of the pathogenic variant site may be 10 or fewer (e.g., between 2 and 10).

In some embodiments of the process 1100, the genomic region may be located on an autosome.

In some embodiments of the process 1100, block 1102 may involve retrieving the phased exome sequence data from a repository of phased whole exome sequences. Additionally, each of the phased whole exome sequences in the repository may correspond to a different individual within a plurality of individuals.

In some embodiments of the process 1100, a pathogenic variant may be associated with the pathogenic variant site. Additionally, a molecular consequence of the pathogenic variant may be a loss of function of an affected transcript.

In some embodiments of the process 1100, the pathogenic variant may include a nonsense mutation, a splice site mutation, an insertion mutation, a deletion mutation, or a missense mutation.

Some embodiments of the process 1100 may further involve receiving a genetic dataset. The genetic dataset may include: (i) a plurality of exome sequences, each exome sequence corresponding to one of a plurality of individuals and (ii) lists of identified pathogenic variant sites, each list corresponding to one of the plurality of exome sequences. The process 1100 may also involve selecting, from the lists of identified pathogenic variant sites, a training pathogenic variant site to train a machine-learned model. Obtaining the phased exome sequence data for the genomic region may include obtaining the exome sequence for the individual from the genetic dataset that corresponds to the list from which the training pathogenic variant site was selected.

Some embodiments of the process 1100 may further involve determining a proband individual with an IBD segment matching the particular phase and within the genomic region. The process 1100 may also involve determining that the individual from the genetic dataset that corresponds to the list from which the training pathogenic variant site was selected shares the IBD segment. Additionally, the process 1100 may involve comparing the pathogenic variant site to the training pathogenic variant site. Further, the process 1100 may involve training the machine-learned model using the IBD segment, the pathogenic variant site, or the phased exome sequence data.

Some embodiments of the process 1100 may further involve determining that a length of the IBD segment exceeds a threshold length for training the machine-learned model. The process 1100 may also involve determining that a minor allele count associated with the pathogenic variant site exceeds a threshold minor allele count for training the machine-learned model.

In some embodiments of the process 1100, the threshold length may be 5 centimorgans and the threshold minor allele count may be 1.

Some embodiments of the process 1100 may further involve determining a first proband individual with a first IBD segment matching the particular phase and within the genomic region. The process 1100 may also involve determining a second proband individual with a second IBD segment matching the particular phase and within the genomic region. Additionally, the process 1100 may involve determining a further individual that shares the first IBD segment and the second IBD segment. Further, the process 1100 may involve generating a notification to the further individual regarding the pathogenic variant site based on the first IBD segment and the second IBD segment.

In some embodiments of the process 1100, generating the notification to the further individual regarding the pathogenic variant site based on the first IBD segment and the second IBD segment may involve generating a first recommendation based on: (i) the first IBD segment; and (ii) a set of quality metrics associated with the first IBD segment. Generating the notification to the further individual regarding the pathogenic variant site based on the first IBD segment and the second IBD segment may also involve generating a second recommendation based on: (i) the second IBD segment; and (ii) a set of quality metrics associated with the second IBD segment. Additionally, generating the notification to the further individual regarding the pathogenic variant site based on the first IBD segment and the second IBD segment may involve determining an overall recommendation by weighting the first recommendation and the second recommendation relative to one another based on the sets of quality metrics associated with the first IBD segment and the second IBD segment.

In some embodiments of the process 1100, the set of quality metrics associated with the first IBD segment may include a read depth associated with a sequencing protocol used to obtain the first IBD segment, an allelic balance associated with the sequencing protocol used to obtain the first IBD segment, or a genotyping quality associated with a dataset from which the statistically phased sequence data was obtained. Such quality metrics may additionally or alternatively be associated with the second IBD segment.

In some embodiments of the process 1100, the first IBD segment may contain the pathogenic variant site, while the second IBD segment may not contain the pathogenic variant site.

In some embodiments of the process 1100, the first IBD segment may contain the pathogenic variant site and the second IBD segment may contain the pathogenic variant site.

### VIII. EXAMPLE TECHNICAL IMPROVEMENTS

Example embodiments described herein provide a technical solution to a technical problem. One technical problem being solved is the computational inefficiency and accuracy limitations in identifying rare, ungenotyped pathogenic variants in large-scale genomic datasets. In practice, identifying these variants in vast datasets can be nearly impossible for a human reviewer (e.g., a clinician or a geneticist), and computationally inefficient methods of algorithmic identification consume excessive processor, memory, network, and power capacity, thereby delaying critical (e.g., timely) medical diagnosis and treatment.

In alternative techniques, such as traditional genetic imputation, systems may rely on population-based reference panels and LD patterns to estimate genotypes. However, these techniques do not effectively resolve rare variants (e.g., variants having minor allele counts between 2 and 10) that are poorly represented in population panels. Moreover, other approaches rely on subjective decisions and experiences of clinicians to manually investigate familial risk, which leads to wildly varying outcomes from clinician to clinician. Still further, other techniques inadequately address the phase inference failures that occur when datasets from different sources (e.g., whole exome sequencing vs. statistical phasing) lack haplotype concordance.

The embodiments herein overcome these limitations. For example, some embodiments implement a nearest-neighbor phase inference algorithm that performs upstream and downstream traversals from a pathogenic variant site to identify non-missing heterozygous sites present in both phased whole exome sequence data and statistically phased sequence data. In this manner, consistent phase assignment and IBD-based imputation can be accomplished in a more accurate and robust fashion without needing to rely on population-level LD structures. This results in several advantages. First, example embodiments significantly improve computational efficiency, allowing for rapid (e.g., real-time) assessments that provide results more quickly and/or using fewer computing resources. Second, example embodiments achieve higher precision for rare variant discovery compared to population-based methods, particularly in the presence of lower minor allele count. Third, example embodiments enable prophylactic treatments and targeted therapies by accurately identifying disease-associated variants (e.g., rare disease-associated variants) in individuals for whom whole exome sequencing has not been performed.

As an example of a prophylactic treatment or targeted therapy, consider an individual with Lynch syndrome (increased risk of hereditary colorectal cancer and/or other forms of cancer). A prophylactic treatment might involve clinical monitoring, such as high-frequency colonoscopies to facilitate the early detection and removal of precancerous polyps. In some cases, daily aspirin may be effective as a form of chemoprevention to reduce the long-term risk of colorectal cancer. For targeted therapies, the presence of specific genetic variants can guide treatment decisions, such as utilizing immunotherapy or checkpoint inhibitors that are particularly effective against the unique biology of certain tumors. By imputing the presence of Lynch syndrome using the techniques described herein (e.g., for an individual for whom whole exome sequencing and targeted assaying of a genotype related to the Lynch syndrome has not been performed), prophylactic treatment and/or targeted therapies may be applied to improve outcomes where, in the absence of imputation techniques, such treatment and/or therapy would not be considered or applied.

Other technical improvements may also flow from these embodiments, and other technical problems may be solved. Thus, this statement of technical improvements is not limiting and instead constitutes examples of advantages that can be realized from the embodiments.

### IX. CLOSING

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step or block that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of non-transitory, computer-readable medium such as a storage device including RAM, ROM, a disk drive, a solid-state drive, or another tangible storage medium.

Moreover, a step or block that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments could include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

What is claimed is:

1. A computer-implemented method comprising:
obtaining phased exome sequence data for a genomic region, wherein the genomic region contains a pathogenic variant site;
obtaining statistically phased sequence data for the genomic region;
traversing the genomic region from the pathogenic variant site in an upstream direction until a first non-missing heterozygous site is present in both the phased exome sequence data and the statistically phased sequence data;
based on haplotypes of the first non-missing heterozygous site from the phased exome sequence data and the statistically phased sequence data, determining an upstream phase;
traversing the genomic region from the pathogenic variant site in a downstream direction until a second non-missing heterozygous site is present in both the phased exome sequence data and the statistically phased sequence data;
based on haplotypes of the second non-missing heterozygous site from the phased exome sequence data and the statistically phased sequence data, determining a downstream phase; and
in response to determining that the upstream phase and the downstream phase are both a particular phase, assigning the particular phase to the genomic region.

2. The computer-implemented method of claim 1, further comprising:
determining a proband individual with an identity-by-descent (IBD) segment matching the particular phase and within the genomic region;
determining a further individual that shares the IBD segment; and
generating a notification to the further individual regarding the pathogenic variant site.

3. The computer-implemented method of claim 2, wherein determining the proband individual comprises determining that the IBD segment has minimum length of 1.5 centimorgans and includes at least 200 single nucleotide polymorphisms.

4. The computer-implemented method of claim 2, further comprising:
determining that a start or an end of the pathogenic variant site exceeds a threshold distance from a start of the IBD segment;
determining that the start or the end of the pathogenic variant site exceeds a threshold distance from an end of the IBD segment;
determining that the start or the end of the pathogenic variant site is within a threshold distance of a center of the IBD segment; or
determining that a length of the IBD segment exceeds a threshold length.

5. The computer-implemented method of claim 2, further comprising applying a targeted therapy to the further individual, wherein the targeted therapy is based on a pathogenic variant associated with the pathogenic variant site, and wherein the targeted therapy comprises a preventative treatment that reduces a disease risk or delays a disease onset associated with the pathogenic variant.

6. The computer-implemented method of claim 1, further comprising:
determining a proband individual with an identity-by-descent (IBD) segment matching the particular phase and within the genomic region;
determining a further individual that shares the IBD segment;
identifying a secondary pathogenic variant site within the IBD segment; and
generating a notification to the further individual regarding the secondary pathogenic variant site.

7. The computer-implemented method of claim 1, further comprising:
determining a first proband individual with a first identity-by-descent (IBD) segment matching the particular phase and within the genomic region;
determining a second proband individual with a second IBD segment matching the particular phase and within the genomic region;
determining a further individual that shares the first IBD segment and the second IBD segment;
selecting, based on a sine-transformed position of the pathogenic variant site within the first IBD segment and a sine-transformed position of the pathogenic variant site within the second IBD segment, the first IBD segment; and
generating a notification to the further individual regarding the pathogenic variant site based on the first proband individual or the first IBD segment.

8. The computer-implemented method of claim 1, wherein the upstream phase is in-phase when the haplotypes of the first non-missing heterozygous site from the phased exome sequence data and the statistically phased sequence data match, wherein the upstream phase is invert-phase when the haplotypes of the first non-missing heterozygous site from the phased exome sequence data and the statistically phased sequence data do not match, wherein the downstream phase is in-phase when the haplotypes of the second non-missing heterozygous site from the phased exome sequence data and the statistically phased sequence data match, and wherein the downstream phase is invert-phase when the haplotypes of the second non-missing heterozygous site from the phased exome sequence data and the statistically phased sequence data do not match.

9. The computer-implemented method of claim 1, wherein:
a minor allele count of the pathogenic variant site is 10 or fewer; or
the genomic region is located on an autosome.

10. The computer-implemented method of claim 1, wherein:
obtaining the phased exome sequence data comprises retrieving the phased exome sequence data from a repository of phased whole exome sequences, wherein each of the phased whole exome sequences in the repository corresponds to a different individual within a plurality of individuals; or
a pathogenic variant is associated with the pathogenic variant site, wherein a molecular consequence of the pathogenic variant is a loss of function of an affected transcript, and wherein the pathogenic variant comprises a nonsense mutation, a splice site mutation, an insertion mutation, a deletion mutation, or a missense mutation.

11. The computer-implemented method of claim 1, further comprising:
receiving a genetic dataset, wherein the genetic dataset comprises:
a plurality of exome sequences, each exome sequence corresponding to one of a plurality of individuals; and
lists of identified pathogenic variant sites, each list corresponding to one of the plurality of exome sequences; and
selecting, from the lists of identified pathogenic variant sites, a training pathogenic variant site to train a machine-learned model,
wherein obtaining the phased exome sequence data for the genomic region comprises obtaining the exome sequence for the individual from the genetic dataset that corresponds to the list from which the training pathogenic variant site was selected.

12. The computer-implemented method of claim 11, further comprising:
determining a proband individual with an identity-by-descent (IBD) segment matching the particular phase and within the genomic region;
determining that the individual from the genetic dataset that corresponds to the list from which the training pathogenic variant site was selected shares the IBD segment;
comparing the pathogenic variant site to the training pathogenic variant site; and
training the machine-learned model using the IBD segment, the pathogenic variant site, or the phased exome sequence data.

13. The computer-implemented method of claim 12, further comprising:
determining that a length of the IBD segment exceeds a threshold length for training the machine-learned model, wherein the threshold length is 5 centimorgans, and wherein the threshold minor allele count is 1; and
determining that a minor allele count associated with the pathogenic variant site exceeds a threshold minor allele count for training the machine-learned model.

14. The computer-implemented method of claim 1, further comprising:
determining a first proband individual with a first identity-by-descent (IBD) segment matching the particular phase and within the genomic region;
determining a second proband individual with a second IBD segment matching the particular phase and within the genomic region;
determining a further individual that shares the first IBD segment and the second IBD segment; and
generating a notification to the further individual regarding the pathogenic variant site based on the first IBD segment and the second IBD segment.

15. The computer-implemented method of claim 14, wherein generating the notification to the further individual regarding the pathogenic variant site based on the first IBD segment and the second IBD segment comprises:
generating a first recommendation based on:
the first IBD segment; and
a set of quality metrics associated with the first IBD segment;
generating a second recommendation based on:
the second IBD segment; and
a set of quality metrics associated with the second IBD segment; and
determining an overall recommendation by weighting the first recommendation and the second recommendation relative to one another based on the sets of quality metrics associated with the first IBD segment and the second IBD segment.

16. The computer-implemented method of claim 15, wherein the set of quality metrics associated with the first IBD segment comprises a read depth associated with a sequencing protocol used to obtain the first IBD segment, an allelic balance associated with the sequencing protocol used to obtain the first IBD segment, or a genotyping quality associated with a dataset from which the statistically phased sequence data was obtained.

17. The computer-implemented method of claim 14,
wherein the first IBD segment contains the pathogenic variant site, and
wherein the second IBD segment does not contain the pathogenic variant site.

18. The computer-implemented method of claim 14,
wherein the first IBD segment contains the pathogenic variant site, and
wherein the second IBD segment contains the pathogenic variant site.

19. A non-transitory, computer-readable medium, having stored thereon program instructions that, upon execution by a computing system, cause the computing system to perform a method comprising:
obtaining phased exome sequence data for a genomic region, wherein the genomic region contains a pathogenic variant site;
obtaining statistically phased sequence data for the genomic region;
traversing the genomic region from the pathogenic variant site in an upstream direction until a first non-missing heterozygous site is present in both the phased exome sequence data and the statistically phased sequence data;
based on haplotypes of the first non-missing heterozygous site from the phased exome sequence data and the statistically phased sequence data, determining an upstream phase;
traversing the genomic region from the pathogenic variant site in a downstream direction until a second non-missing heterozygous site is present in both the phased exome sequence data and the statistically phased sequence data;
based on haplotypes of the second non-missing heterozygous site from the phased exome sequence data and the statistically phased sequence data, determining a downstream phase; and
in response to determining that the upstream phase and the downstream phase are both a particular phase, assigning the particular phase to the genomic region.

20. A system comprising:
one or more processors; and
memory, containing program instructions that, upon execution by the one or more processors, cause the system to perform a method comprising:
obtaining phased exome sequence data for a genomic region, wherein the genomic region contains a pathogenic variant site;
obtaining statistically phased sequence data for the genomic region;
traversing the genomic region from the pathogenic variant site in an upstream direction until a first non-missing heterozygous site is present in both the phased exome sequence data and the statistically phased sequence data;
based on haplotypes of the first non-missing heterozygous site from the phased exome sequence data and the statistically phased sequence data, determining an upstream phase;
traversing the genomic region from the pathogenic variant site in a downstream direction until a second non-missing heterozygous site is present in both the phased exome sequence data and the statistically phased sequence data;
based on haplotypes of the second non-missing heterozygous site from the phased exome sequence data and the statistically phased sequence data, determining a downstream phase; and
in response to determining that the upstream phase and the downstream phase are both a particular phase, assigning the particular phase to the genomic region.
